(19) United States
(12) Patent Application Publication
(10) Pub. No.: US 2026/0207131 A1
(43) Pub. Date: Jul. 23, 2026

(54) TECHNIQUES FOR ANALYZING NON-COMPLIANT WEARABLE DEVICE DATA

(71) Applicant: Eli Lilly and Company, Indianapolis, IN (US)

(72) Inventors: Jian YANG, Carmel, IN (US); Hui ZHANG, Carmel, IN (US)

(21) Appl. No.: 19/139,087

(22) PCT Filed: Dec. 14, 2023
(86) PCT No.: PCT/US2023/083979

§ 371 Date: Jun. 13, 2025

Related U.S. Application Data
(60) Provisional application No. 63/387,547, filed on Dec. 15, 2022.

(51) Int. Cl. A61B 5/00; A61B 5/11

(52) U.S. Cl. CPC A61B 5/4833; A61B 5/1118; A61B 5/7221

(57) ABSTRACT

A method is provided for determining a quality of at least some data acquired by a plurality of sensors. The method includes: receiving, from a plurality of sensors associated with a plurality of subjects, data acquired by the plurality of sensors during an acquisition period, wherein the data is indicative of signals associated with bodies of the plurality of subjects; dividing the acquisition period into a plurality of subperiods; aggregating at least some of the data into a plurality of subsets of data, wherein each of the plurality of subsets of data comprises data acquired during a different subperiod of the plurality of subperiods; determining, for each particular subperiod of the plurality of subperiods, a quality of the subset of data acquired during said particular subperiod; and outputting an indication of the determined quality of at least one subset of data of the plurality of subsets of data.

### BACKGROUND OF INVENTION

Sensors are used to collect data about the human body. A sensor may detect a physiological or biokinetic signal, such as an acceleration, ECG signal, temperature, or glucose levels, for example. The signal can be used to monitor overall health of the patient, monitor variation of a particular parameter, and detect disease onset, among other applications.

### SUMMARY OF INVENTION

According to an exemplary embodiment of the present disclosure, a method is provided. The method includes: using one or more processors to perform: receiving, from a plurality of actigraphy sensors associated with a plurality of subjects, data acquired by the plurality of actigraphy sensors during an acquisition period, wherein the data is indicative of signals associated with bodily movement of the plurality of subjects, the receiving comprising: receiving, from a first actigraphy sensor of the plurality of actigraphy sensors associated with a first subject of the plurality of subjects, first data acquired by the first actigraphy sensor, wherein the first data is indicative of signals associated with bodily movements of the first subject; dividing the acquisition period into a plurality of subperiods; aggregating at least some of the data into a plurality of subsets of data, the at least some of the data including the first data, wherein each of the plurality of subsets of data comprises data acquired during a different subperiod of the plurality of subperiods; determining, for each particular subperiod of the plurality of subperiods, a quality of the subset of data acquired during the particular subperiod, the determining comprising: determining whether the first subject of the plurality of subjects was compliant in using the first actigraphy sensor of the plurality of actigraphy sensors during the particular subperiod; and outputting an indication of the determined quality of at least one subset of data of the plurality of subsets of data.

According to another embodiment of the present disclosure, a system is provided. The system includes a memory storing instructions, and a processor configured to execute the instructions to perform a method. The method includes: receiving, from a plurality of actigraphy sensors associated with a plurality of subjects, data acquired by the plurality of actigraphy sensors during an acquisition period, wherein the data is indicative of signals associated with bodily movement of the plurality of subjects, the receiving comprising: receiving, from a first actigraphy sensor of the plurality of actigraphy sensors associated with a first subject of the plurality of subjects, first data acquired by the first actigraphy sensor, wherein the first data is indicative of signals associated with bodily movements of the first subject; dividing the acquisition period into a plurality of subperiods; aggregating at least some of the data into a plurality of subsets of data, the at least some of the data including the first data, wherein each of the plurality of subsets of data comprises data acquired during a different subperiod of the plurality of subperiods; determining, for each particular subperiod of the plurality of subperiods, a quality of the subset of data acquired during the particular subperiod, the determining comprising: determining whether the first subject of the plurality of subjects was compliant in using the first actigraphy sensor of the plurality of actigraphy sensors during the particular subperiod; and outputting an indication of the determined quality of at least one subset of data of the plurality of subsets of data.

According to another embodiment of the present disclosure, a non-transitory computer-readable media is provided. The non-transitory computer-readable media includes instructions that, when executed by one or more processors on a computing device, are operable to cause the one or more processors to execute a method. The method includes: receiving, from a plurality of actigraphy sensors associated with a plurality of subjects, data acquired by the plurality of actigraphy sensors during an acquisition period, wherein the data is indicative of signals associated with bodily movement of the plurality of subjects, the receiving comprising: receiving, from a first actigraphy sensor of the plurality of actigraphy sensors associated with a first subject of the plurality of subjects, first data acquired by the first actigraphy sensor, wherein the first data is indicative of signals associated with bodily movements of the first subject; dividing the acquisition period into a plurality of subperiods; aggregating at least some of the data into a plurality of subsets of data, the at least some of the data including the first data, wherein each of the plurality of subsets of data comprises data acquired during a different subperiod of the plurality of subperiods; determining, for each particular subperiod of the plurality of subperiods, a quality of the subset of data acquired during the particular subperiod, the determining comprising: determining whether the first subject of the plurality of subjects was compliant in using the first actigraphy sensor of the plurality of actigraphy sensors during the particular subperiod; and outputting an indication of the determined quality of at least one subset of data of the plurality of subsets of data.

### BRIEF DESCRIPTION OF DRAWINGS

Additional embodiments of this disclosure, as well as features and advantages thereof, will become more apparent by reference to the description herein taken in conjunction with the accompanying drawings. The components in the figures are not necessarily to scale. Moreover, in the figures, like-referenced numerals designate corresponding parts throughout the different views.

FIG. 1A and FIG. 1B are diagrams depicting an illustrative method for determining a quality of data acquired by a sensor, according to some embodiments.

FIG. 2A and FIG. 2B are block diagrams depicting an exemplary system for determining a quality of data acquired by a sensor, according to some embodiments.

FIG. 3A is a flowchart showing an exemplary computerized method for determining a quality of data acquired by a sensor, according to some embodiments.

FIG. 3B is an illustrative plot showing data acquired by a sensor during an acquisition period, according to some embodiments.

FIG. 3C is an example graphical user interface (GUI) indicating quality of data acquired by a sensor, according to some embodiments.

FIG. 4A is a block diagram showing subjects participating in one or more clinical studies, according to some embodiments.

FIG. 4B is an example report indicating compliance of subjects who are participating in a clinical study in using their sensors, according to some embodiments.

FIG. 4C is an example report indicating compliance of subjects who are associated with a particular clinical study site in using their sensors, according to some embodiments.

FIG. 4D is an example report indicating compliance of an individual subject in using their sensor(s), according to some embodiments.

FIG. 5 is a flowchart showing an exemplary computerized method for determining a pattern of non-compliance of a subject in using a sensor, according to some embodiments.

FIG. 6A is an example report indicating the degree to which a first subject was compliant in using a sensor during each hour of multiple days of an acquisition period, according to some embodiments.

FIG. 6B is an example table indicating whether or not the first subject was compliant in using the sensor during each hour of the multiple days of the acquisition period, according to some embodiments.

FIG. 6C is an example table indicating an hour of the day that the first subject was frequently noncompliant in using the sensor over the multiple days, according to some embodiments.

FIG. 6D is an example table indicating days during which the first subject was frequently noncompliant in using the sensor, accordingly to some embodiments.

FIG. 6E is an example plot showing a trend in the first subject's compliance in using the sensor, according to some embodiments.

FIG. 7A is an example report indicating the degree to which a second subject was compliant in using a sensor during each hour of multiple days of an acquisition period, according to some embodiments.

FIG. 7B is an example table indicating whether or not the second subject was compliant in using the sensor during each hour of the multiple days of the acquisition period, according to some embodiments.

FIG. 7C is an example table indicating an hour of the day that the second subject was frequently noncompliant in using the sensor over the multiple days, according to some embodiments.

FIG. 7D is an example table indicating days during which the second subject was frequently noncompliant in using the sensor, accordingly to some embodiments.

FIG. 7E is an example plot showing a trend in the second subject's compliance in using the sensor, according to some embodiments.

### DETAILED DESCRIPTION OF INVENTION

For the purposes of promoting an understanding of the principles of the present disclosure, reference will now be made to the embodiments illustrated in the drawings, and specific language will be used to describe the same. It will nevertheless be understood that no limitation of the scope of the invention is thereby intended.

Provided herein are techniques for determining a quality of data acquired by one or more sensors. The data can be acquired during an acquisition period. For example, a sensor may be configured to detect a signal associated with a subject's body during the acquisition period. In some embodiments, the techniques include (a) dividing the acquisition period into multiple subperiods, (b) aggregating the data acquired by the sensor into multiple subsets of data, and (c) determining a quality of each subset of data. In some embodiments, determining a quality of a subset of data includes determining whether a subject was using the sensor during a particular subperiod.

Connected clinical trials allow physiological signals to be collected by sensors for research and development of digital biomarkers (dBMs), which are objective, quantifiable physiological, and behavioral characteristics that can explain and predict health-related outcomes that cannot be captured during in-clinic visits or through patient questionnaires.

A computer-based platform can be configured to ingest and process data from sensors, in real-time, for clinical trials. The inventors have recognized that developing and discovering novel dBMs is hypothesis-driven research, and the process involves handling unprecedented quantities of digital data generated from digital technologies. As one nonlimiting example, with a sampling frequency of 50 Hz, over 4 million 3-axial data points are collected from an accelerometer for a single patient to understand the one patient's daily activities. Accessing, processing, understanding, and visualizing such large amounts of data is resource intensive and inefficient.

Furthermore, effective results depend on trusted and understood data collected from sensors and digital devices. However, connected clinical trials are often conducted under free-living conditions, in which a subject wears a sensor device on a best effort basis as the subject attempts to comply with instructions communicated or provided to the subject during study enrollment. Such conditions can introduce issues such as missing data or invalid data when subjects do not wear, or incorrectly wear, the device. Because missing and invalid data do not accurately reflect the physical condition of the subject, it can introduce inaccuracies into dBMs that are derived from such data. For example, if a subject does not wear a sensor for a significant portion of the day, resulting acceleration data may suggest that the subject is inactive for that portion of the day, which may not, in fact, be an accurate reflection of the subject's activity.

Accordingly, the inventors have developed techniques which address the above-described limitations of conventional biosensing techniques. The techniques can automatically analyze the quality of the obtained data and provide indications of data quality (e.g., including graphical displays). In some embodiments, the techniques include determining a quality of data acquired by one or more sensors worn by users. As used herein, a “quality” of data acquired by such sensors can refer to a suitability of such acquired data for drawing scientific and/or medical conclusions. The quality can be determined for the data during an acquisition period, such as a time when the user was tasked to wear the sensor for collecting data for one or more clinical trials. For example, determining a quality of the data may include determining whether a subject was using the sensor properly, whether the device was (or was not) charged, determining errors (e.g., in connectivity, synchronization with a data source, etc.), whether the data includes nonsense values (e.g., indicative of a damaged device), whether the data includes values outside a dynamic output range of the sensor, or any other suitable quality metric. The data can be analyzed as part of a computer-based platform that ingests and processes data from user-worn sensors. In some embodiments, the quality of the data may be used to determine whether the data should be used in downstream analysis, such as for monitoring the subject's health, developing biomarkers, or for any other suitable application. The data can be analyzed in real time as part of the platform.

The quality of the data can be evaluated in various ways according to the techniques described herein. The data can be evaluated based on the trial that the data is being collected for. For example, users may be instructed to wear the sensor while sleeping, while exercising, etc. The techniques can aggregate the data and evaluate quality over time (e.g., over five minutes, a day, a week, etc.) and/or space (e.g., different geographical sites or locations, such as by city, by state, by country, etc.).

In some embodiments, determining a quality of data acquired by the sensor during an acquisition period includes (a) dividing the acquisition period into subperiods, (b) aggregating the data into subsets of data, where each subset of data includes data acquired during a different subperiod, and (c) determining a quality of each subset of data. In some embodiments, aggregating data in this manner allows for an efficient and accurate evaluation of the data without analyzing each data point acquired by each sensor during the acquisition period. For example, in a large-scale clinical study, a single data subset may represent data acquired by thousands of sensors during a particular time period of the clinical study, allowing for a single determination of the quality of that data. As described herein, despite the data representing massive amounts of information, among other benefits, the determination of quality can be used to understand the quality of the dataset at a more holistic level. Such holistic quality information can be used to develop clinical trials, including to more accurately determine biomarker values and predict health outcomes.

In some embodiments, the techniques provide for viewing and analyzing reports of the quality analysis of the data. The data can be viewed and filtered in different ways, such as by calendar days and/or aligned trial days. The quality can be provided based on a time period, such as based on the number of minutes per day (e.g., the number of minutes of good quality data, or as a percentage out of the number of minutes of a day). In some embodiments, reports can be generated or viewed on a patient level, a visit level, and/or a time period level (e.g., daily). Accordingly, the data can be aggregated in various ways (e.g., based on trial, location, and/or user) and reported at different levels (e.g., daily, visit, and/or user) to provide in-depth and custom data analysis, which can improve the development of clinical trials.

In particular, the quality analysis can be used to improve the development and/or running of clinical trials by informing when data should be acquired and/or used for clinical trials. This can help to avoid acquiring or using data during times when subjects typically are not compliant in using the sensor. Being able to analyze when to use or acquire data can result in one or more improvements to the operation of clinical trials. For example, it can increase the efficiency of clinical trials because less time will be required to filter through the massive amounts of acquired data to distinguish between data representing a biological signal and bad or unusable data, such as data representing the sensor sitting unused by the subject or data generated by a faulty or malfunctioning sensor. As another example, it can improve the accuracy of metrics determined using the acquired data because the acquired data represents biological signals (e.g., bodily movement) as opposed to signals representing nonuse of the sensor by the subject. The quality analysis can additionally or alternatively be used to improve the accuracy of biomarker values that are determined using the acquired data. In particular, the quality analysis can be used to identify low-quality data and to exclude the low-quality data from the determination of the biomarker values. Excluding low-quality data, such as that which represents sensor malfunctioning or nonuse by the subject, and including only data that represents the bodily signals of the subject improves the accuracy of the biomarker values determined using such data.

While users can attempt to manually review quality analysis reports to eyeball patterns in the data, the inventors have recognized that this is often inefficient and infeasible, and such an approach may also risk missing (important) patterns. Consider, for example, a clinical trial with hundreds or thousands of participants, each of whom is using a sensor over the duration of days, weeks, months, or even longer. Such a large number of participants can result in a massive amount of data, especially when coupled with a long the trial duration. Attempting to eyeball reports generated for each individual participant would be both time intensive and inaccurate given the sheer volume of data and the presence of subtle patterns in the data. Additionally, the human reviewers would themselves likely need to trigger next steps for addressing the patterns of noncompliance, introducing additional inefficiencies (and possible errors) into the overall process.

Accordingly, the inventors have developed techniques for automatically determining patterns of subject noncompliance (or compliance) when using a sensor, and summarizing such patterns of noncompliance for consumption by a user or clinical trial sponsor. For example, the techniques may include automatically identifying certain periods of time (e.g., day(s), hour(s) of the day, week(s), etc.) that a subject is frequently noncompliant in using a sensor. Such techniques have several practical applications, as discussed further below.

For example, in some embodiments, the techniques can be used to efficiently enforce subject compliance in using a sensor (e.g., during a clinical trial). For example, the determined patterns of noncompliance can be used to automatically trigger a notification to the subject (e.g., at the time of detection and/or during the times when the subject is found to be frequently noncompliant). The notification may be in the form of an automatic text message, phone call, e-mail, push notification, and/or any other suitable type of notification. The notification may prompt the subject to use the sensor during the times when the subject has been found to be typically noncompliant. By automatically reviewing for patterns of noncompliance and prompting subjects to use their sensors in real-time during a clinical trial, the techniques can be used to obtain more useable data throughout the trial, thereby improving the overall quality of data that is collected.

Additionally, or alternatively, the techniques for determining patterns of noncompliance can be used to understand the functioning of the sensor and/or the device comprising the sensor (e.g., a wearable device that houses the sensor). For example, regular periods of noncompliance in using the sensor may correlate to times that a subject is charging the device. This information can be used to understand characteristics of the device such as average battery life and the time needed to recharge the battery. Additionally, or alternatively, this information can be used to design a second device and/or second clinical trial that has improved characteristics. For example, the second device may be deployed to participants, and used to determine patterns of noncompliance in using the second device. The patterns of noncompliance in using the second device may be compared to the patterns of noncompliance in using the first device to measure a degree to which the second device is an improvement over the first. For example, if the regular periods of noncompliance in using the second device are shorter than the regular periods of noncompliance in using the first device, this may indicate that the second device has a shorter recharge time.

While various embodiments have been described, it will be apparent to those of ordinary skill in the art that many more embodiments and implementations are possible. Accordingly, the embodiments described herein are examples, not the only possible embodiments and implementations. Furthermore, the advantages described above are not necessarily the only advantages, and it is not necessarily expected that all of the described advantages will be achieved with every embodiment.

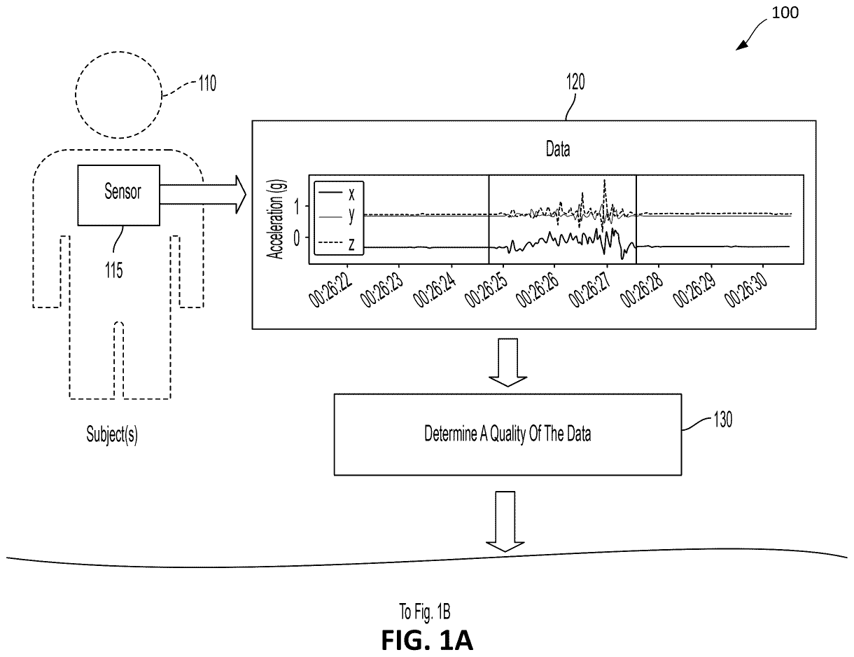

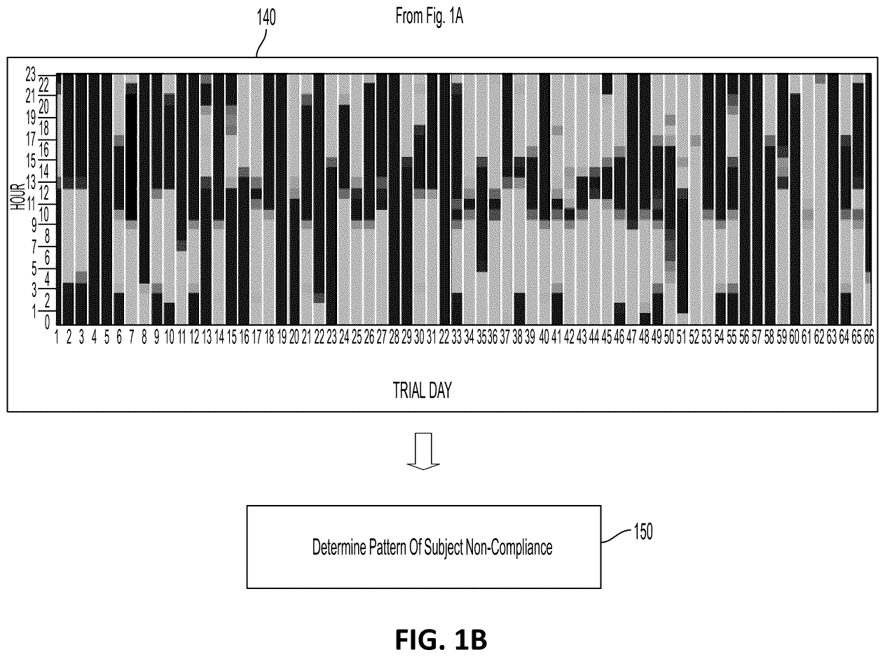

FIGS. 1A and 1B are diagrams depicting an illustrative method 100 for determining a quality of data 120 acquired by sensor 115, according to some embodiments.

As shown, illustrative method 100 includes receiving data 120 from sensor 115 associated with subject 110. As described herein, in some embodiments, the data 120 is received by one or more processors configured to perform one or more steps of illustrative method 100. For example, the one or more processors may be configured to determine a quality of the received data 120 (e.g., step 130) and output an indication 140 of the determined quality.

In some embodiments, the sensor 115 is worn by or implanted in the body of the subject 110. For example, the sensor 115 may be included in a device that takes the form of an accessory (e.g., a watch, glasses, jewelry, etc.) worn by the subject, a body-mounted device (e.g., a patch attached to the subject's skin), a device embedded in the subject's clothing, an ear-worn device, an implantable device, or any other suitable type of device, as aspects of the technology described herein are not limited to a particular type of device.

In some embodiments, the sensor 115 is configured to detect a signal associated with the body of the subject 110. As nonlimiting examples, the sensor 115 may include an actigraphy sensor, an electrocardiogram (ECG) sensor, a blood glucose sensor, a thermometer, an electromyogram (EMG) sensor, a tissue oximeter, a pulse oximeter, a respiration rate sensor, a heart rate sensor, a skin perspiration sensor, a motion sensor, an accelerometer, a position sensor, or any other suitable sensor, as aspects of the technology described herein are not limited in this respect.

In some embodiments, the sensor 115 is configured to acquire the signal during an acquisition period. For example, the sensor 115 may be configured to continuously acquire the signal during the acquisition period or periodically (e.g., cyclically or intermittently) acquire the signal during the acquisition period. In some embodiments, the acquisition period is of any suitable duration, as aspects of the technology described herein are not limited in this respect. For example, the acquisition period may be on the order of seconds, minutes, hours, days, weeks, months, or years.

In some embodiments, data 120 is indicative of the signal detected by the sensor 115. For example, the data 120 may include the raw signal data acquired by the sensor 115. Additionally, or alternatively, the data 120 may include data that has been processed using any suitable signal processing techniques, as aspects of the technology described herein are not limited to any particular signal processing technique. As a nonlimiting example, as shown in FIG. 1A, the data 120 may include data indicative of an acceleration of the sensor 115.

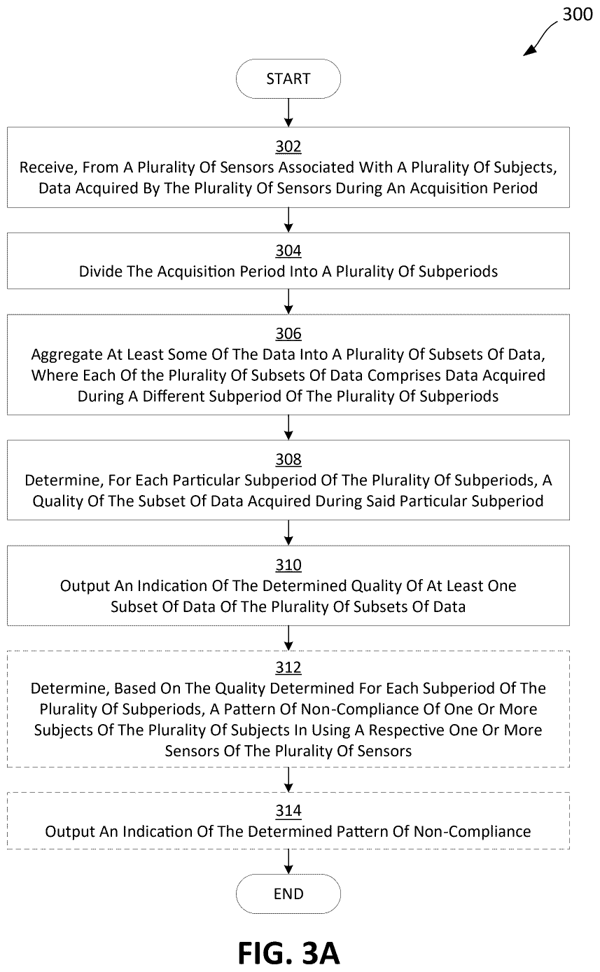

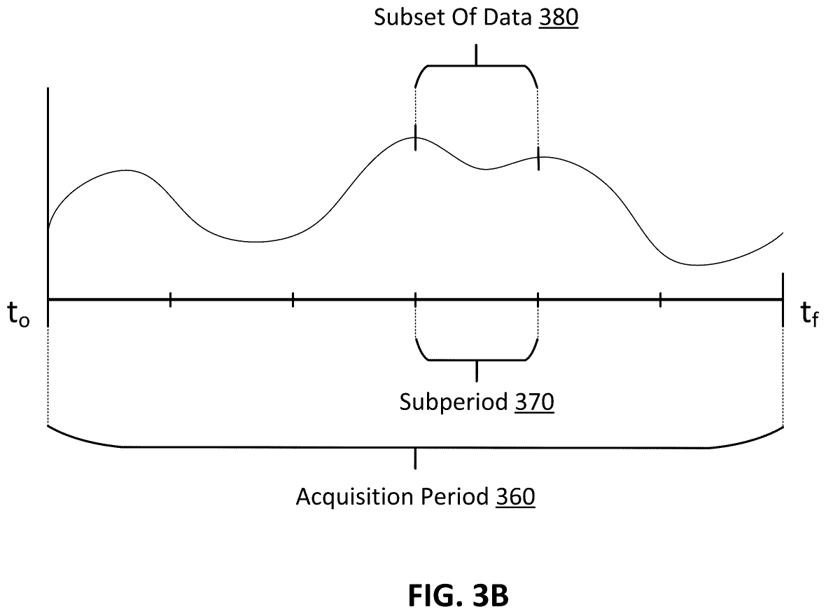

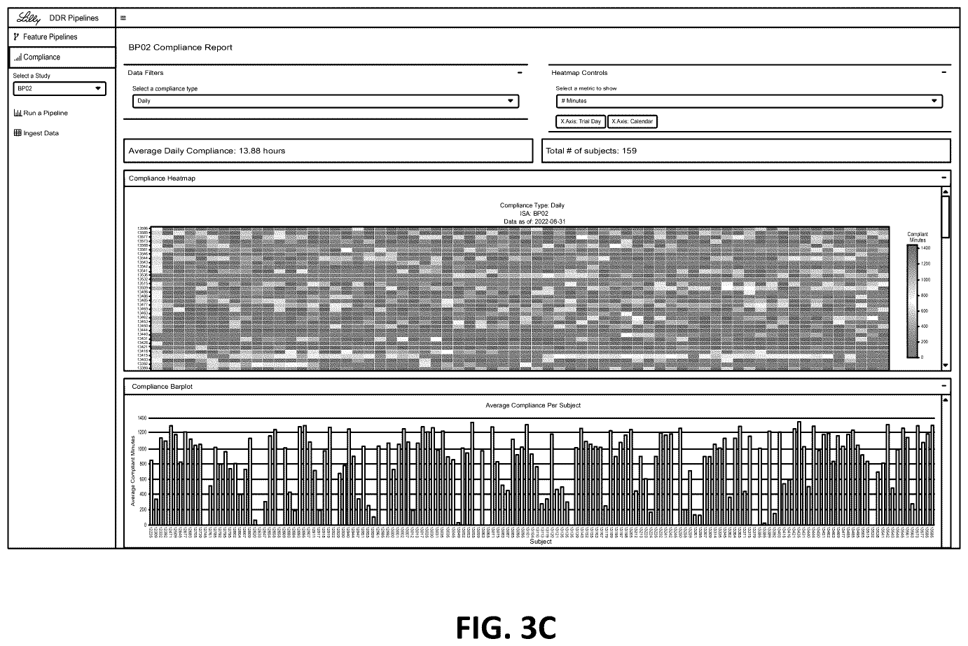

In some embodiments, at step 130 of illustrative method 100, one or more processors determine a quality of the received data 120. For example, determining the quality of the received data may include determining whether the subject 110 was compliant in using the sensor 115 during the acquisition period, determining whether the sensor 115 malfunctioned during the acquisition period, or determining any other suitable metric indicative of quality of the data, as aspects of the technology described herein are not limited in this respect. Example techniques for determining a quality of such data are described herein including at least with respect to FIGS. 3A-3C.

In some embodiments, after determining the quality of the data at step 130, the one or more processors output an indication 140 of the determined quality. For example, the output may indicate whether the subject 110 was compliant in using the sensor 115 during the acquisition period, whether the sensor 115 malfunctioned during the acquisition period, and/or any other suitable indication of the determined quality of the data, as aspects of the technology described herein are not limited in this respect. FIG. 1B shows an example output 140 of the illustrative method 100. The example output 140 identifies particular hours on particular days during which the subject was compliant in using the sensor. In the example depicted in FIG. 1B, each column is associated with a separate trial day of a clinical trial, and each row is associated with a different hour of a 24-hour day. Each cell in the depicted matrix is given a color or shading that corresponds to the quality of data collected from a particular subject 110 during that associated day and hour. For instance, a lighter color may indicate good quality data was collected, while a darker color may indicate that lower quality data was collected.

In some embodiments, outputting the indication 140 of the determined quality includes generating a graphical user interface that includes the indication, generating a report that includes the indication, transmitting the indication to another device, storing the indication, or outputting the indication according to any other suitable techniques, as aspects of the technology described herein are not limited in this respect.

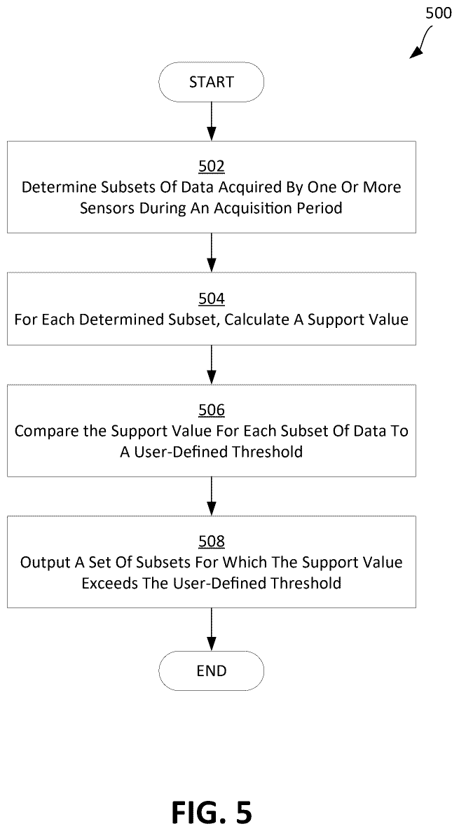

Additionally, or alternatively, in some embodiments, the determined quality of data is used to determine, at act 150, a pattern of subject non-compliance (or compliance) in using the sensor 115. In some embodiments, determining the pattern of subject non-compliance includes identifying one or more days during the acquisition period during which the subject was frequently noncompliant in using the sensor. For example, this may include determining that the subject was relatively less compliant in using the sensor for certain days, such as the first, middle, and/or last day(s) of the acquisition period (e.g., during the start and/or end of the acquisition period, or during one or more days when the patient could not wear the sensor). Additionally, or alternatively, in some embodiments, determining the pattern of subject non-compliance includes identifying one or more hours during the day that the subject was relatively less compliant in using the sensor over the acquisition period. For example, this may include determining that the subject was generally less compliant in using the sensor in the early hour(s) of the day, during hour(s) in the middle of the day, and/or during the late hour(s) of the day. Example techniques for determining patterns of subject noncompliance are described herein including at least with respect to FIG. 3A, FIG. 5 and FIGS. 6A-7E.

In some embodiments, the determined pattern of subject noncompliance may be used to provide specific instructions to the subject regarding how to use the sensor. For example, the determined pattern of subject noncompliance may be used to prompt the subject to use the sensor during the day(s) and/or time(s) when they have historically been noncompliant. Additionally, or alternatively, the determined pattern of subject noncompliance may be used by another user (e.g., an administrator of a clinical trial, a healthcare provider, etc.) to instruct the subject to use the sensor and/or to design or improve future clinical trials. Further, as another example, the determined pattern of subject noncompliance can be used to determine data that is not used as part of a clinical trial. Correcting and/or adjusting sensor usage can directly improve the data that is collected by the sensor, which will in turn improve the accuracy of determining biomarkers and predicting health outcomes using said data.

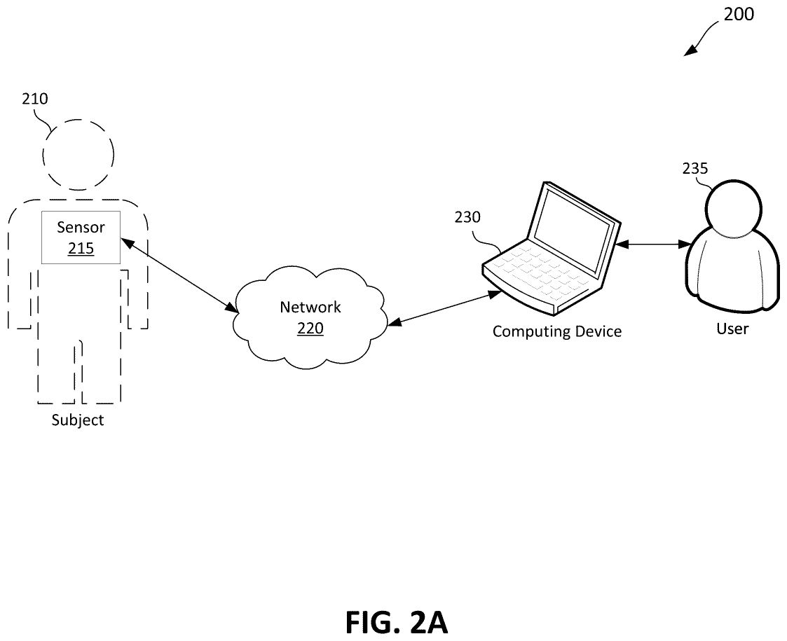

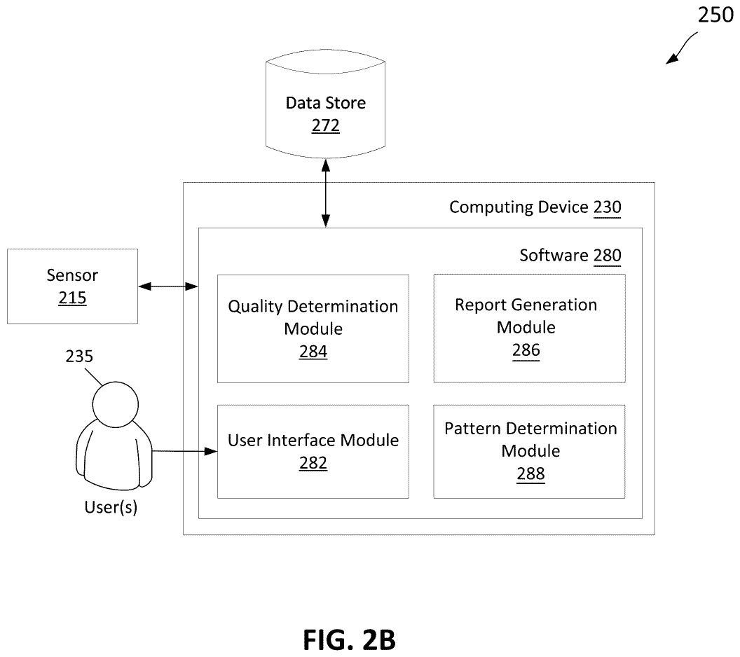

FIG. 2A is a block diagram depicting an exemplary system 200 for determining a quality of data acquired by a sensor, according to some embodiments. As shown, system 200 includes sensor 215, computing device 230, and network 220. It should be appreciated, however, that a system for determining a quality of data acquired by a sensor may include one or more additional or alternative components, as aspects of the technology described herein are not limited in this respect.

In some embodiments, sensor 215 (e.g., sensor 115 shown in FIG. 1A) is associated with subject 210. In some embodiments, sensor 215 includes one or more sensors that are associated with a single subject. For example, the one or more sensors may be used to detect different biokinetic and/or physiological signals associated with the subject's body. Additionally, or alternatively, the sensor 215 may include multiple sensors associated with multiple subjects. For example, the sensors may be used to monitor multiple subjects during a clinical study.

In some embodiments, after acquiring a signal associated with the subject's body, the sensor 215 transmits data indicative of the acquired signal to the computing device 230 via network 220. In some embodiments, the sensor 215 transmits the data in real-time. For example, the sensor 215 may transmit the data in response to detecting the signal. Additionally, or alternatively, in some embodiments, the sensor 215 transmits the data within a threshold time (e.g., within seconds, minutes, hours, etc.) of detecting the signal. Additionally, or alternatively, in some embodiments, the sensor 215 transmits the data in response to a request to transmit the data. For example, the sensor 215 may receive a request from computing device 230 and/or via a user interface associated with the sensor 215.

In some embodiments, the data indicative of the acquired signal includes raw (e.g., unprocessed) signal data. Additionally, or alternatively, the data indicative of the detected signal includes signal data that has been processed using any suitable signal processing techniques, as aspects of the technology described herein are not limited in this respect. For example, the signal may be processed to ensure that it is suitable for transmission via network 220.

Network 220 may be or include a wide area network (e.g., the Internet), a local area network (e.g., a corporate Internet), and/or any other suitable type of network. Sensor 215 and/or computing device 230 may connect to the network 220 using one or more wired links, one or more wireless links, and/or any suitable combination thereof. Accordingly, the network 220 may be, for example, a hard-wired network (e.g., a local area network within a healthcare facility), a wireless network (e.g., connected over Wi-Fi and/or cellular networks), a cloud-based computing network, or any combination thereof.

In some embodiments, computing device 230 is used to determine a quality of the data received from the sensor 215. For example, determining the quality of the received data may include determining whether the subject 210 was compliant in using the sensor 215 during the acquisition period, determining whether the sensor 215 malfunctioned during the acquisition period, or determining any other suitable metric indicative of quality of the data, as aspects of the technology described herein are not limited in this respect. Example techniques for determining a quality of such data are described herein including at least with respect to FIGS. 3A-3C.

In some embodiments, the computing device 230 includes one or multiple computing devices. When computing device 230 includes multiple computing devices, the device(s) may be physically co-located (e.g., in a single room) or distributed across multiple physical locations. In some embodiments, computing device 230 may be part of a cloud computing infrastructure. In some embodiments, one or more computing devices 230 may be co-located in a facility operated by an entity.

In some embodiments, the computing device 230 is associated with a user 235. For example, the user 235 may include a researcher and/or healthcare professional. Such a researcher and/or healthcare professional may monitor the data acquired by the sensor 215 to determine whether the subject 210 is being compliant in using the sensor 215, to monitor the health of the subject 210, and/or to determine whether the sensor 215 is functioning properly. Additionally, or alternatively, in some embodiments, the user 235 includes subject 210. For example, the subject 210 may monitor his or her health using the sensor data.

In some embodiments, the computing device 230 is further configured to receive input from user 235 and/or generate an output. For example, as described herein including at least with respect to FIG. 2B, the computing device 230 may include a user interface configured to receive user input and/or display an output to the user 235. As described herein, in some embodiments, the user input indicates one or more criteria for determining a quality of the data from sensor 215. Additionally, or alternatively, in some embodiments, the user input is used to generate an output according to the preferences of the user 235.

FIG. 2B is a block diagram of computing device 230 shown in FIG. 2A, according to some embodiments. In some embodiments, computing device 230 includes software 280 configured to perform various functions with respect to data received from sensor 215. In some embodiments, software 280 includes a plurality of modules. A module may include processor-executable instructions that, when executed by at least one computer hardware processor, cause the at least one computer hardware processor to perform the function(s) of the module. Such modules are sometimes referred to herein as “software modules.” As described herein, the system of FIG. 2A can be used to provide a computer-based platform to collect data for clinical trials, for example.

As shown in FIG. 2B, in some embodiments, software 280 includes a user interface module 282, a quality determination module 284, a report generation module 286, and a pattern determination module 288.

In some embodiments, the user interface module 282 is a graphical user interface (GUI), a text-based user interface, and/or any other suitable type of interface through which a user may provide input and/or receive output. For example, in some embodiments, the user interface module 282 may be a webpage or web application accessible through an Internet browser. In some embodiments, the user interface module 282 may be a GUI of a software application (app) executing on the user's mobile device. In some embodiments, the user interface module 282 may include a number of selectable elements through which the user may interact. For example, the user interface module 282 may include dropdown lists, checkboxes, text fields, or any other suitable elements, as aspects of the technology described herein are not limited in this respect.

In some embodiments, the quality determination module 284 processes data acquired by a sensor 215 to determine a quality of the data. For example, the quality determination module 284 may determine whether a subject was compliant in using sensor 215 during an acquisition period, whether the sensor 215 malfunctioned during the acquisition period, and/or any other suitable metric indicative of the quality of the data acquired by the sensor 215.

In some embodiments, processing the data includes (a) dividing the acquisition period into multiple subperiods, (b) aggregating the data into multiple subsets of data, where each subset of data includes data acquired during a different subperiod, and (d) determining a quality of a subset of the data that includes data acquired during a particular subperiod. For example, the subperiods may be determined based on user input provided via the user interface module 282. Additionally, or alternatively, the quality of a subset of data may be determined based on one or more criteria provided via user interface module 282 and/or stored in data store 272. Techniques for processing data to determine a quality of the data are described herein including at least with respect to FIGS. 3A-3C.

In some embodiments, the quality determination module 284 obtains the sensor data from sensor 215 and/or from a data store 272 configured to store data from the sensor 215. For example, software 280 may include one or more interface modules (not shown), such as a sensor interface module and/or a data store interface module. The sensor interface module may be configured to obtain (either pull or be provided) data from the sensor 215. The data store interface module may be configured to obtain (either pull or be provided) data from the data store 272. The data may be provided via network 220.

In some embodiments, data store 272 includes one or more data stores configures to store data from sensor 215, data associated with a clinical study, and/or data obtained via user interface module 282. The data store 272 may include any suitable data store, such as a flat file, a database, a multi-file, or data storage of any suitable type, as aspects of the technology described herein are not limited to any particular type of data store.

In some embodiments, the data associated with a clinical study may include identification information for subjects participating in the clinical study, information about different clinical study sites, and/or any other suitable information relating to conducting a clinical study, as aspects of the technology described herein are not limited in this respect.

In some embodiments, the data obtained via the user interface module 282 includes criteria for determining a quality of data acquired by the sensor 215. For example, as described herein, the criteria may indicate how to divide the acquisition period (e.g., a duration of a subperiod of the acquisition period), the type and/or amount of data to be processed for determining the quality of the data, the type of quality metric to be determined, one or more threshold values with which to compare the data, and/or any other suitable types of criteria, as aspects of the technology described herein are not limited in this respect.

In some embodiments, the pattern determination module 288 is configured to determine a pattern of subject noncompliance based on the quality of data determined by quality determination module 284. In some embodiments, the pattern determination module 288 obtains the quality data from the quality determination module 284 and/or data store 272. In some embodiments, the pattern determination module 288 is configured to perform frequent itemset mining to identify one or more periods of time (e.g., one or more days, one or more hours during the day, etc.) during which the subject is not compliant in using the sensor. Techniques for determining a pattern of subject noncompliance are described herein including at least with respect to FIG. 3A and FIGS. 6A-7E.

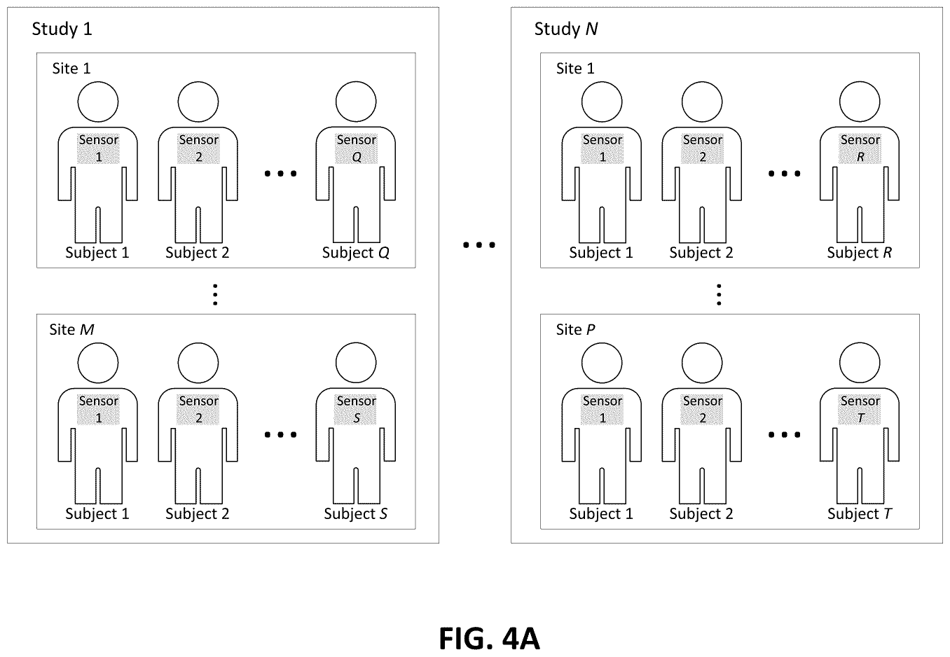

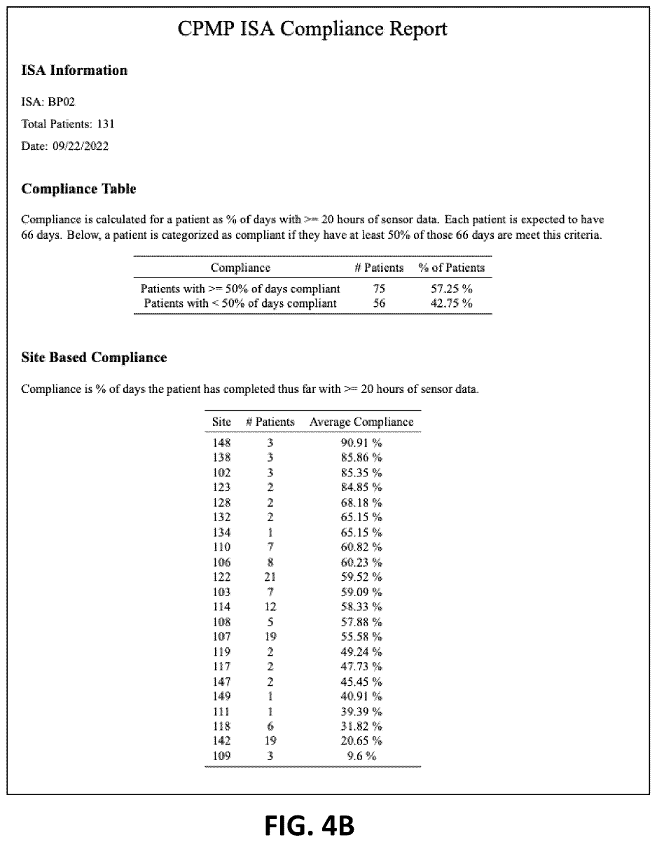

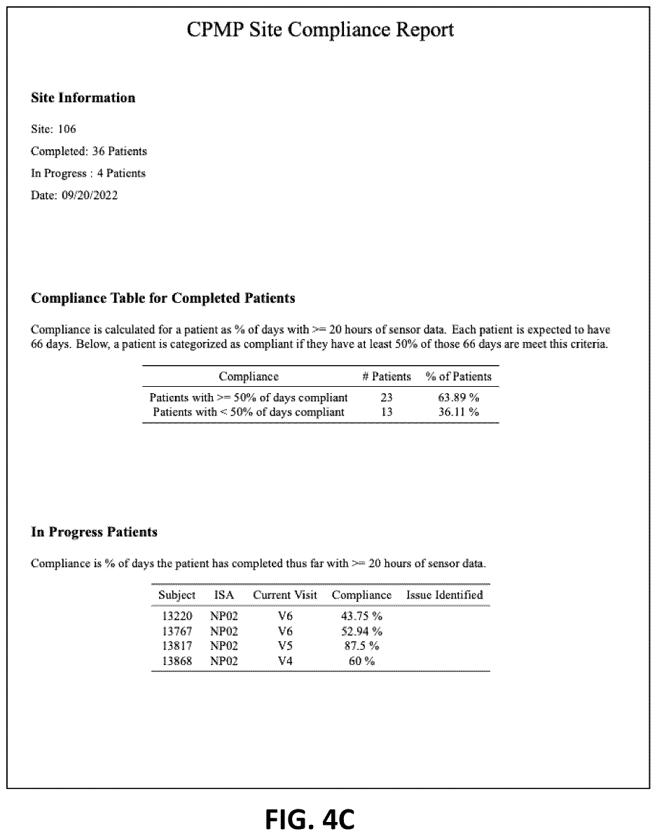

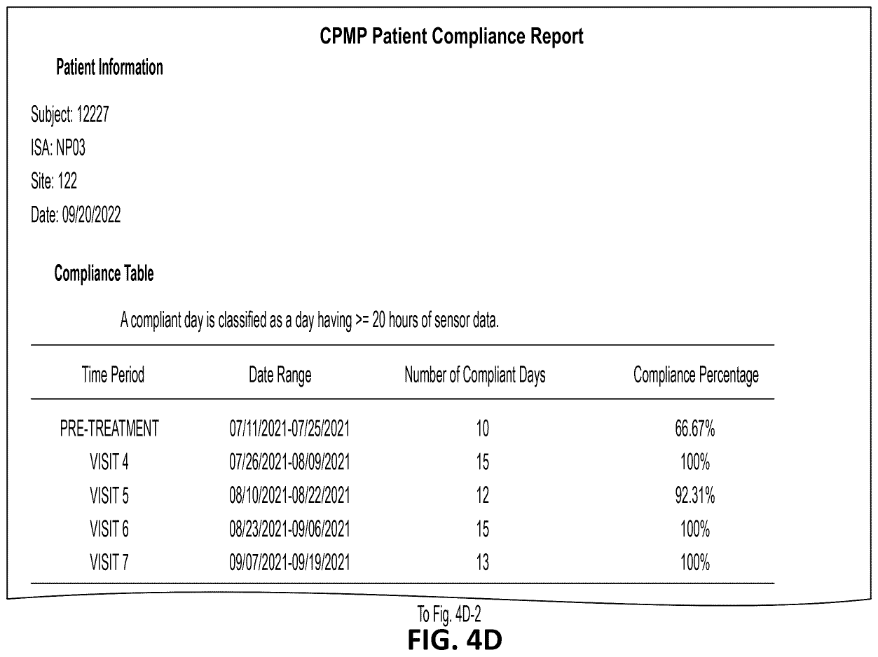

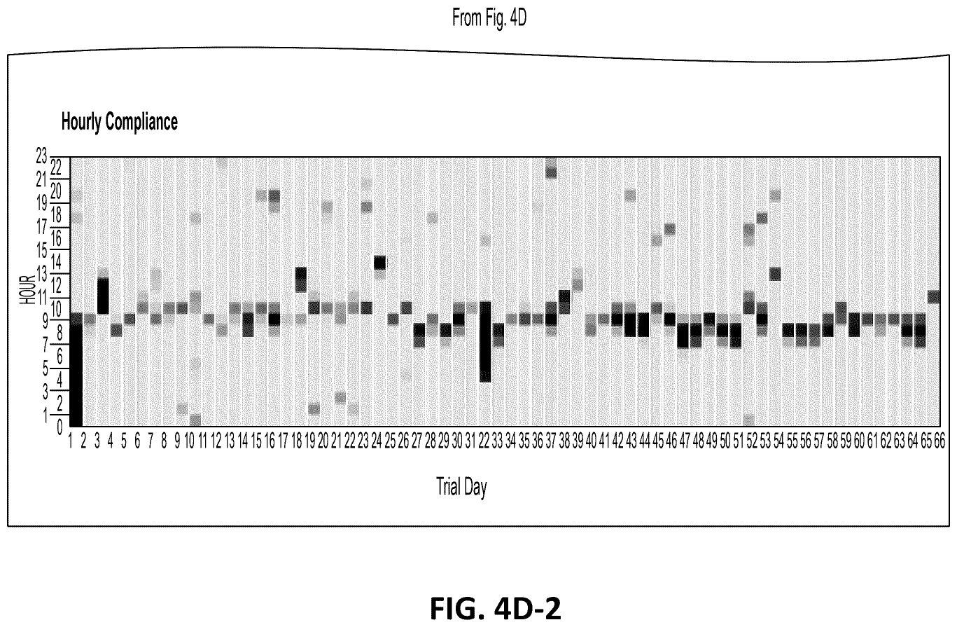

In some embodiments, the report generation module 286 generates a report indicating the quality of the data determined using the quality determination module 284. For example, the report generation module 286 may generate a report indicating the compliance of subjects in using their sensors 215. Additionally, or alternatively, the report generation module 286 may generate a report indicating whether the sensor 215 malfunctioned during an acquisition period. Additionally, or alternatively, the report generation module 286 may generate a report indicating a pattern of subject noncompliance, as determined by the pattern determination module 288. Additionally, or alternatively, the reportion generation module 286 may generate a report including instructions to a user (e.g., the subject) regarding how to use the sensor. It should be appreciated, however, that the report generation module 286 may generate any suitable type of report, as aspects of the technology are not limited in this respect. Example reports are described herein including at least with respect to FIGS. 3C and 4B-4D.

In some embodiments, reports generated by the report generation module 286 are output using any suitable output techniques, such as, for example, outputting the report through the user interface module 282, storing the report in the data store 272, and/or transmitting the report to another device.

FIG. 3A is a flowchart showing an exemplary method 300 for determining a quality of data acquired by a sensor, according to some embodiments. Method 300 may be implemented on any suitable processor, such as computing device 230 shown in FIGS. 2A and 2B, for example, and/or any other suitable processor, as aspects of the technology described herein are not limited in this respect.

At step 302, the processor receives from a plurality of sensors associated with a plurality of subjects, data acquired by the plurality of sensors during an acquisition period. For example, the data acquired by the plurality of sensors may be indicative of a biokinetic and/or physiological signal associated with the subject's body. The sensors may include sensor 115 shown in FIG. 1A, sensor 215 shown in FIGS. 2A and 2B, an actigraphy sensor, an electrocardiogram (ECG) sensor, a blood glucose sensor, a thermometer, an electromyogram (EMG) sensor, a tissue oximeter, a pulse oximeter, a respiration rate sensor, a heart rate sensor, a skin perspiration sensor, a motion sensor, an accelerometer, a position sensor, or any other suitable sensor, as aspects of the technology described herein are not limited in this respect.

In some embodiments, the plurality of subjects includes two or more subjects each using a respective one or more sensors. For example, each subject may use one or more sensors to monitor his or her individual health. Additionally, or alternatively, each subject may use the one or more sensors as part of a clinical study. For example, the plurality of subjects may be participating in different clinical studies, and/or in same clinical study. Subjects participating the same clinical study may be associated with different clinical study sites and/or the same clinical study site. An example clinical study structure is described herein including at least with respect to FIG. 4A.

In some embodiments, receiving the data includes receiving the data in real time (e.g., as the sensor acquires the data), within a threshold time of the sensor acquiring the data (e.g., within seconds, within minutes, within hours, withing days, etc.), in response to a request by the processor, and/or in response to user input requesting transmittal of the data. In some embodiments, the processor receives the data via a network, such as network 220 shown in FIG. 2A

In some embodiments, the acquisition period is the time period during which the data was acquired by the sensor. The acquisition period may be of any suitable duration, as aspects of the technology are not limited in this respect. For example, FIG. 3B shows an example acquisition period 360 starting at an initial time, to, and ending at a final time, tr. The initial and final time may be specified using any suitable techniques, as aspects of the technology are not limited in this respect. For example, when the data is received in real-time, the acquisition period may include the time elapsed between an initial time and a current time. Additionally, or alternatively, the acquisition period may be specified by a user and/or by a computing device (e.g., a user may provide user input indicating the initial and final times of the acquisition period).

At step 304, the processor divides the acquisition period into a plurality of subperiods. In some embodiments, a subperiod of the acquisition period refers to a time period within the acquisition period that is of a shorter duration than the duration of the acquisition period. For example, as shown in FIG. 3B, acquisition period 360 is divided into multiple subperiods, including subperiod 370. The subperiod may be of any suitable duration within the acquisition period, as aspects of the technology are not limited in this respect. As a nonlimiting example, an acquisition period of one month may be divided into subperiods of a week's duration, subperiods of an hour's duration, subperiods of a minute's duration, subperiods of a second's duration, or subperiods of any other suitable duration.

In some embodiments, the acquisition period is divided into the plurality of subperiods based on user input. For example, a user (e.g., user 235 shown in FIGS. 2A and 2B) may indicate the duration of the subperiod. As a nonlimiting example, a user, such as a researcher, may be interested in determining a quality of the data on an hour-by-hour basis, and therefore may indicate that the acquisition period is to be divided into subperiods having a duration one hour. In this example, based on the user input, the processor may divide the acquisition period into subperiods having a duration of one hour.

At step 306, the processor aggregates at least some of the data into a plurality of subsets of data. For example, a subset of data may include data that was acquired during a particular subperiod. For example, as shown in FIG. 3B, the subset of data 380 includes data that was acquired during the subperiod 370 of the acquisition period 360. In some embodiments, a subset of data includes data acquired by a single sensor during a particular subperiod. Additionally, or alternatively, in some embodiments, a subset of data includes data acquired by multiple sensors during a particular subperiod.

At step 308, the processor determines, for each particular subperiod of the plurality of subperiods, a quality of the subset of data acquired during said particular subperiod. For example, with reference to FIG. 3B, the processor may determine a quality of the subset of data 380. While various examples of determining a quality of a subset of data are described herein, it should be appreciated that any suitable techniques may be performed to determine the quality of the data, as aspects of the technology described herein are not limited to any particular technique for determining the quality of a subset of data.

In some embodiments, determining a quality of a subset of data includes determining whether one or more subjects were compliant in using one or more sensors during the particular subperiod. For example, where the sensor(s) include accelerometer(s), determining whether the subject(s) were compliant in using the sensor(s) during a particular subperiod may include evaluating the acceleration data acquired by the accelerometer(s) during that particular subperiod. This may involve, in some embodiments, evaluating the standard deviation and/or the value range of the data included in the subset of data acquired during the particular subperiod. For example, if the standard deviation of the acceleration data acquired during a particular subperiod is less than a threshold value (e.g., a compliance threshold), this may indicate that the subject(s) were not using the sensor(s) during the particular subperiod. In some embodiments, the threshold value includes any suitable value such as, for example, a value that is indicated by a user via user input, as aspects of the technology are not limited in this respect. Examples of determining whether a subject is compliant in using a sensor during a particular subperiod of an acquisition period are described herein including at least in the section “Example 4-Example Techniques for Determining Data Quality.”

In some embodiments, determining a quality of a subset of data includes determining whether one or more sensors malfunctioned during the particular subperiod. For example, in some embodiments, determining whether the sensor(s) malfunctioned includes determining whether the subset of data acquired during the particular subperiod includes values that are outside the output range of the sensor(s). For example, if the data includes a value that exceeds a maximum output value of the sensor(s), or if the data includes a value that is less than a minimum output value of the sensor(s), this may indicate that some of the data is invalid. In some embodiments, if the subset of data includes values outside of the output range, such values may be removed and excluded from further analysis. Additionally, or alternatively, the processor may output an indication that the subset of data includes invalid values.

In some embodiments, determining whether the one or more sensors malfunctioned includes determining a proportion of values included in the subset of data that are at or near (e.g., within a threshold value) a maximum or minimum output value of the sensor(s). If a threshold proportion of values included in the subset of data are at or near a maximum or minimum output value of the sensor(s), this may indicate that “clipping” is occurring, and that the data is invalid. For example, if at least 40%, at least 45%, at least 50%, at least 55%, at least 60%, at least 65%, at least 70%, at least 75%, at least 80%, at least 90%, or at least 100% of the values included in a subset of data are at or near the maximum (or minimum) output value of the sensor(s), the processor may determine that clipping occurred, and that the sensor(s) malfunctioned during the particular subperiod. Examples of determining whether the sensor malfunctioned during a particular subperiod are described herein including at least with respect to the section “Example 4—Example Techniques for Determining Data Quality.”

Furthermore, in some embodiments, determining a quality of a subset of data may include aggregating quality metrics derived from a plurality of time periods within the particular subperiod. For example, if the particular subperiod is one day, determining a quality of a subset of data corresponding to that one day may first comprise determining a quality of data (e.g., using any of the aforementioned methods, such as determining whether the sensor malfunctioned, detecting “clipping”, etc.) of multiple time periods within that day. Each time period within the particular subperiod may span one minute, five minutes, ten minutes, one hour, or any other suitable time period. In some embodiments, each such time period within a particular subperiod may be referred to as an “epoch”. Determining a quality of data for each epoch may comprise determining a binary indication regarding whether the data collected within that epoch was of good quality or bad quality or may comprise a value that indicates in more granular fashion the value of data collected within that epoch (e.g., a numerical scale from 0 to 10). Once an indicator of quality of data for each epoch within a particular subperiod has been determined, these indicators may be aggregated to determine a single indication of quality of data for the entire particular subperiod (e.g., for the entire day). For example, this aggregation may comprise computing a mean or median average of the indicators for each epoch within the particular subperiod. Alternatively or in addition, this aggregation may comprise an indication of the number of epochs within the particular subperiod that had “good” data, e.g., data that fit certain predetermined threshold criteria. Alternatively or in addition, this aggregation may comprise an indication of whether the number of epochs with good data is above or below a certain predetermined threshold (e.g., whether the subject collected at least six hours' worth of good data within the day, or whether at least 50% of the epochs had good data). Although the example presented above corresponded to the scenario where the particular subperiod corresponds to one day, and epochs that correspond to one, five, ten, or sixty minutes, it should be understood that subperiods and epochs may be set to different lengths. For example, subperiods may correspond to five days, one week, two weeks, or one month, and an epoch may correspond to a 12-hour, 24-hour, or 2-3 day period.

At step 310, the processor outputs an indication of the determined quality of at least one subset of data of the plurality of subsets of data. In some embodiments, the processor outputs the indication using any suitable output techniques, as aspects of the technology described herein are not limited to any particular technique. For example, the processor may output the indication of the quality via a user interface, such as the user interface module 282 shown in FIG. 2B, the example GUI shown in FIG. 3C, or through any other suitable user inface. Additionally, or alternatively, in some embodiments, outputting the indication includes storing the indication. For example, the indication may be stored in a data store, such as data store 272 shown in FIG. 2B. Additionally, or alternatively, outputting the indication includes transmitting the indication to another device, such as another computing device, a printer, or any other suitable device for viewing, processing, and/or storing the indication.

The indication of the quality of data may improve the accuracy of determining biomarker values and predicting health outcomes. For example, the indication of quality may be used to exclude poor-quality data from such biomarker calculations or health outcome predictions. For example, the poor-quality data may reflect data acquired during times when the subject was not using the sensor and/or data acquired during times when the sensor was malfunctioning. Data acquired during these times does not accurately reflect the biological signals (e.g., bodily movement) of the subject and therefore would contribute to inaccurate biomarker estimations and health outcome predictions which are based on biological signal data.

Additionally, or alternatively, the indication of the quality of the data may be used to instruct the subject regarding how to use the sensor and/or address sensor malfunction issues. Addressing such issues will help to improve the quality of the data acquired in the future. This, in turn, will improve the accuracy of biomarker value determinations and health outcome predictions because such determinations and predictions will be based on greater amounts of high quality data acquired during the acquisition period.

At step 312, the processor optionally determines, based on the quality determined for each subperiod at step 308, a pattern of noncompliance of one or more subjects in using a respective one or more sensors of the plurality of sensors. For example, in some embodiments, the output of step 308 includes, for each subperiod, an indication of whether or not the subject was compliant in using the sensor during the particular subperiod. The indications of subject compliance may be used, at step 312, to determine the pattern of non-compliance over the acquisition period.

In some embodiments, determining the pattern of compliance includes (a) determining subsets of the acquisition period to evaluate for patterns of noncompliance, (b) for each determined subset, calculating a support value based on the quality determined at act 308, (c) comparing the support value for each subset of data to a threshold (e.g., a user-defined threshold), and (d) identifying a set of the subsets for which the support value exceeds the threshold. Example techniques for determining a pattern of noncompliance are described herein including at least with respect to FIG. 5-FIG. 7E.

In some embodiments, the pattern of noncompliance is indicative of period(s) of time during which the subject was frequently noncompliant in using the sensor during the acquisition period. For example, the pattern of noncompliance may be indicative of a set of one or more days during the acquisition period during which the subject was frequently noncompliant in using the sensor. Additionally, or alternatively, the pattern of noncompliance may be indicative of a set of one or more hours of the day during the acquisition period during which the subject was frequently noncompliant in using the senor. Additionally, or alternatively, the pattern of noncompliance may be indicative of any other suitable period(s) of time during which the subject was frequently noncompliant in using the sensor during the acquisition period, as aspects of the technology described herein are not limited in this respect.

At step 314, the processor optionally outputs an indication of the determined pattern of noncompliance. In some embodiments, the indication may be output in conjunction with or independent from the indication of quality output at step 310. In some embodiments, the processor outputs the indication using any suitable output techniques, as aspects of the technology described herein are not limited to any particular technique. For example, the processor may output the indication of the pattern via a user interface, such as the user interface module 282 shown in FIG. 2B or through any other suitable user inface. Additionally, or alternatively, in some embodiments, outputting the indication includes storing the indication. For example, the indication may be stored in a data store, such as data store 272 shown in FIG. 2B. Additionally, or alternatively, outputting the indication includes transmitting the indication to another device, such as another computing device, a printer, or any other suitable device for viewing, processing, and/or storing the indication.

In some embodiments, the indication of the determined pattern of noncompliance includes instructions for the subject. For example, the instructions may prompt the subject to use the sensor during one or more times that the pattern indicates the subject has not been compliant in using the sensor in the past. Providing instructions to the subject may improve the quality of the data obtained during future uses of the sensor because the data will no longer include as much “missing” data during periods of noncompliance. The instructions may be provided directly to the subject (e.g., via a notification or report) or indirectly to the subject (e.g., through a healthcare provider or administrator of a clinical trial). For example, the instructions may be provided in the form of a text message, phone call, e-mail, push notification, and/or any other suitable type of notification, as aspects of the technology described herein are not limited in this respect.

In some embodiments, the instructions may be provided to the subject automatically upon detection of the pattern of noncompliance. Additionally, or alternatively, the instructions may be provided to the subject after a threshold period of time has elapsed since the pattern of noncompliance was initially detected. For example, the techniques may include initially detecting a pattern of noncompliance and if, after the threshold period of time has elapsed, the subject is still exhibiting the pattern of noncompliance, providing the instructions to the subject. The threshold period of time may include any suitable threshold, as aspects of the technology described herein are not limited in this respect. For example, the threshold period of time may be at least one day, at least five days, at least one week, at least two weeks, at least one month, at least two months, between six hours and one year, between one day and six months, between five days and two months, between one week and one month, or any other suitable threshold period of time.

Additionally, or alternatively, in some embodiments, the techniques include providing instructions in different formats dependent on the subject's historical pattern(s) of noncompliance. For example, in some embodiments, if a pattern of noncompliance has been detected for a subject fewer than a threshold number of times, instructions may be provided to the subject via an automatic notification (e.g., an automatic text message, e-mail, push notification, etc.). If a pattern of noncompliance has been detected for a subject greater than or equal to the threshold number of times, this may indicate that a greater level of intervention is needed, and the instructions may be provided to the subject in a different format. For example, in addition to, or as alternative of, sending an automatic notification to the subject, an automatic notification may be sent to a researcher and/or administrator, such that said researcher and/or administrator may arrange for a person (e.g., healthcare provider, clinical trial administrator, etc.) to directly contact the subject to provide them with instructions for using the sensor and/or for soliciting input as to why the subject is not using the sensor.

Additionally, or alternatively, in some embodiments, outputting the indication of the determined pattern of noncompliance at act 314 may include outputting an indication of whether or not the subject improved in using the sensor relative to the subject's past use. For example, a first pattern of noncompliance may have been previously determined for the subject. The first pattern of compliance may be compared to the pattern of compliance determined at step 312 of process 300 (e.g., the second pattern of noncompliance) to determine a degree of improvement (or non-improvement or deterioration) in the subject's use of the sensor since the first pattern of noncompliance was determined. If the results of the comparison indicate that the subject has not improved in using the sensor, an intervention may be triggered (e.g., instructions may be provided to the subject to use the sensor).

In some embodiments, method 300 may be performed to determine a first pattern of noncompliance of one or more subjects in using a sensor included in a first device. In some embodiments, the first pattern of noncompliance may be used to determine characteristics of the first device. For example, regular periods of noncompliance may be indicative of times when a subject was charging the first device. In some embodiments, these periods of noncompliance may be used to infer the battery life and/or time needed to charge the first device.

The determined characteristics of the first device may be used, in some embodiments, to design a second device. For example, the second device may be designed to improve the characteristics of the first device. The second device may be distributed to one or more subjects (e.g., the same subjects that used the first device or different subjects). Method 300 may be repeated to determine a second pattern of noncompliance of one or more subjects in using the sensor included in the second device. In some embodiments, the second pattern of noncompliance may be compared to the first pattern of noncompliance. The results of the comparing may be used to determine whether one or more characteristics of the second device are an improvement over the characteristics of the first device. For example, if the second pattern of noncompliance indicates that the regular periods of noncompliance are less frequent or of shorter duration, this may indicate that the second device has a longer battery life and/or shorter charging time, and/or is more convenient for users to charge and use in a compliant manner.

FIG. 3C is an example graphical user interface (GUI) indicating quality of data acquired by a sensor, according to some embodiments. As shown, the GUI displays a compliance heatmap and a compliance bar plot. The compliance heatmap includes a row for each of multiple subjects, and a column for each day of an acquisition period. The compliance heatmap indicates, for each subject, for each day, the proportion of minutes of a day that the subject used their sensor(s) using different colors and/or shading; for example, cool colors (e.g., blues and/or greens) may indicate high compliance, while warm colors (e.g., reds and/or oranges) may indicate low compliance. The compliance bar plot indicates, for each subject, the average number of minutes each day that they used their sensor(s) during the acquisition period. In some embodiments, a user of the GUI may interact with GUI by hovering the cursor over different elements shown on the GUI.

Additionally, or alternatively, in some embodiments, the user may use the drop-down menus to manipulate the indication of the determined quality of the data. For example, the user may use the “Compliance Type” drop-down menu to select different types of reports to be generated. The “Compliance Type” drop-down menu may include options for generating reports based on different subperiod durations. For example, selecting a “Weekly,” “Bi-Weekly,” or “Monthly” type report may change the heatmap such that each column corresponds to one week, two weeks, or one month, respectively.

Additionally, or alternatively, the “Compliance Type” drop-down menu may include options for generating reports for different levels of a clinical study (e.g., by individual, by clinical study site(s), by study, etc.). For example, selecting a “Site Level” type report may change the heatmap such that each row corresponds not to an individual, but to a site. Such a heatmap would show the aggregated compliance for all subjects within the indicated site. Such an aggregation may comprise computing a mean or median average of the indicators of quality determined for each subject associated with the site. Alternatively or in addition, this aggregation may comprise an indication of the number of subjects associated with the site that had “good” data, e.g., data that fit certain predetermined threshold criteria. Alternatively or in addition, this aggregation may comprise an indication of whether the number of subjects with good data is above or below a certain predetermined threshold. In some embodiments, selecting a “Study Level” type report may change the heatmap such that each row corresponds not to an individual subject or site, but to a whole clinical study. Such a heatmap would show the aggregated compliance for all subjects within the indicated study. Such an aggregation may comprise computing a mean or median average of the indicators of quality determined for each subject associated with the study. Alternatively or in addition, this aggregation may comprise an indication of the number of subjects associated with the study that had “good” data, e.g., data that fit certain predetermined threshold criteria. Alternatively or in addition, this aggregation may comprise an indication of whether the number of subjects with good data is above or below a certain predetermined threshold.

The example GUI shown in FIG. 3C also includes a drop-down menu for selecting a metric to show. In some embodiments, this drop-down can be manipulated to allow the user to choose how to evaluate the compliance of one or more subjects and/or the quality of the data. For example, the drop-down menu may allow a user to select a threshold with which to compare acceleration data to determine compliance. If the standard deviation of the acceleration data acquired during a particular subperiod is less than the selected threshold value, this may indicate that the subject(s) were not using the sensor(s) during the particular subperiod. Accordingly, the heatmap may provide an indication as to whether the subject was compliant during the particular subperiod. Additionally, or alternatively, the dropdown menu may allow a user to select a clipping threshold. The techniques described herein may be used to (a) determine a proportion of values included in the subset of data that are at or near a maximum or minimum output value of the sensor(s), and (b) compare the determined proportion to the selected clipping threshold. If the proportion exceeds the clipping threshold, this may indicate that “clipping” is occurring, and that the data is invalid. Accordingly, the heatmap may provide an indication as to whether clipping was occurring.

Additionally, or alternatively, in some embodiments, the processor may output the indication via one or more report documents. For example, FIGS. 4B-4D are example reports that may be in PDF format or any other suitable format, as aspects of the technology described herein are not limited in this respect.

Researchers and healthcare professionals frequently conduct large-scale clinical studies (e.g., clinical trials) to find new and better ways to detect, understand, and treat medical conditions. This involves the collection, organization, and analysis of massive amounts of data. For example, multiple (e.g., tens, hundreds, thousands, etc.) of subjects may participate in a clinical study of several clinical studies. Data may be acquired from each of the subjects (e.g., using one or more sensors) continuously or periodically (e.g., cyclically, or intermittently) over long durations of time (e.g., over days, weeks, months, or years).

For example, as shown in FIG. 4A, each clinical study (e.g., clinical studies 1-N) may have multiple sites. A site may refer to a location, such as a hospital, research center, or medical institution, for example, which is participating in the clinical study. For example, as shown in FIG. 4A, each clinical study has a respective number of participating clinical sites (e.g., study 1 has M sites and study N has P sites.) Additionally, one or more subjects may be participating in each clinical study through the available sites. For example, Q subjects are participating though site 1 of study 1, S subjects are participating through site M of study 1, R subjects are participating through site 1 of study N, and T subjects are participating through site P of study N. Data is collected from each subject participating in each clinical study.

In some embodiments, the techniques described herein are used to determine a quality of data at the level of a clinical study. For example, the techniques may be used to determine the compliance of the subjects 1-Q and subjects 1-S in using the sensors 1-Q and sensors 1-S during each subperiod (e.g., a minute, an hour, a day, a week, etc.) of an acquisition period (e.g., an hour, a day, a week, a month, etc.) of clinical study 1. In such an embodiment, the data acquired by each of the sensors 1-Q and sensors 1-S is processed according to the techniques described herein to determine the quality of the data.

Additionally, or alternatively, in some embodiments, the techniques described herein are used to determine a quality of data at the level of a site of a clinical study. For example, the techniques may be used to determine the compliance of subjects 1-Q, at site 1, in using sensors 1-Q during each subperiod (e.g., a minute, an hour, a day, a week, etc.) of an acquisition period (e.g., an hour, a day, a week, a month, etc.) of clinical study 1. In such an embodiment, the data acquired by each of the sensors 1-Q is processed according to the techniques described herein to determine the quality of the data.

Additionally, or alternatively, in some embodiments, the techniques described herein are used to determine a quality of data at the level of an individual participating in a clinical study. For example, the techniques may be used to determine a compliance of subject Q in using sensor Q during each subperiod (e.g., a minute, an hour, a day, a week, etc.) of an acquisition period (e.g., an hour, a day, a week, a month, etc.) of clinical study 1. In such an embodiment, the data acquired by sensor Q is processed according to the techniques described herein to determine the quality of the data.

FIG. 4B is an example report indicating study-level compliance of subjects who participated in a clinical study, according to some embodiments. A study-level report may contain metrics displaying overall enrollment and compliance on a site level. These may allow a clinical trial team to gauge the progress of a specific study easily, i.e., the number of patients who have completed their time in the study and the number of patients still in progress. As indicated in the report, the data was acquired for 131 patients during an acquisition period of 66 days. The acquisition period was divided into subperiods of one hour.

The sensor data from each of the 131 patients was aggregated into subsets of data, each of which included sensor data acquired by the sensors during a particular hour of the acquisition period. The data was processed to determine a quality of each subset of data during each particular hour. The results are shown in the Compliance Table. As shown, 75 patients used their sensor for more than (or equal to) 20 hours a day for more than (or equal to) 50% of the total number of days of the acquisition period.

Additionally, for each site, the sensor data from patients associated with the particular site was aggregated into subsets of data, each of which included data acquired by the sensors during a particular hour of the acquisition period. For example, for site 148, the sensor data from three patients was aggregated into a plurality of subsets of data. For each site, the data was processed to determine a quality of each subset of data during each particular hour. The results are shown in the Site Based Compliance Table. As shown, on average, the three patients at site 148 used their sensors for more than 20 hours a day for 90.91% of the days of the acquisition period.

FIG. 4C is an example report indicating compliance of subjects associated with a particular clinical study site, according to some embodiments. In some embodiments, generating reports based on sites allows clinical teams to efficiently identify which sites may be experiencing issues regarding low compliance across their assigned patients. In some embodiments, site reports contain information for overall performance, with specifics for patients that may fall below a set compliance threshold. The patients with low compliance may be labeled with a potential issue-such as low compliance during the nighttime. The potential issues may be derived from the hourly compliance for that patient. From here, sites can identify which of their patients contribute most to low compliance and attempt to resolve the issues linked to the low compliance.

As indicated in the report of FIG. 4C, 40 patients are associated with the clinical site, 36 of whom have completed the clinical study and 4 of whom are still in the process of completing the clinical study. The acquisition period was 66 days and was divided into subperiods of one hour.

The sensor data from each of the 36 patients who completed the clinical study was aggregated into subsets of data, each of which included sensor data acquired by the sensors during a particular hour of the acquisition period. The data was processed to determine a quality of each subset of data during each particular hour. The results are shown in the Compliance Table for Completed Patients. As shown, 23 of the completed patients used their sensor for more than (or equal to) 20 hours a day for more than (or equal to) 50% of the total number of days of the acquisition period.

Additionally, for each patient, the sensor data from the patient was aggregated into subsets of data, each of which included data acquired by the sensor during a particular hour of the acquisition period. For example, for patient 13220, the sensor data from the patient was aggregated into a plurality of subsets of data. For each patient, the data was processed to determine a quality of the subset of data acquired during each particular hour of the acquisition period. The results are shown in the In Progress Patient Table. As shown, on average, patient 13220 used their sensor for more than 20 hours a day for 43.75% of the days of the acquisition period.

FIG. 4D is an example report indicating compliance of an individual subject in using their sensor(s), according to some embodiments. In some embodiments, reports on a patient level can give insight into their specific patterns of device wearing. In these reports, in some embodiments, the number of visits, compliant days within each visit, and compliance percentage per visit may be displayed. In addition, in some embodiments, an hourly compliance heatmap may be visible, allowing for further understanding of when patients wear their devices across the study duration.

As indicated in the example report of FIG. 4D, the acquisition period was 66 days and was divided into subperiods of one hour.

The sensor data from the patient was aggregated into subsets of data, each of which included data acquired by the sensor during a particular hour of the acquisition period. The data was processed to determine a quality of each subset of data acquired during each particular hour of the acquisition period. The results of the analysis are shown in the Compliance Table and in the Hourly Compliance Heatmap.

The Compliance Table shows, for each of multiple date ranges, the number and percentage of days during the particular date range that the subject was compliant in using the sensor. The subject was considered to be compliant if they used their sensor for more than (or equal to) 20 hours of a day. For example, as shown, during the pre-treatment, the subject used the sensor for more than (or equal to) 20 hours a day for 10 days (or 66.67%) of the pre-treatment time period.

The Hourly Compliance Heatmap indicates the specific hours during which the subject was using or not using the sensor during each day of the acquisition period. Darker shading indicates poor compliance, while lighter shading indicates good compliance.

FIG. 5 is a flowchart showing an exemplary computerized method 500 for determining a pattern of non-compliance of a subject in using a sensor, according to some embodiments. Method 500 may be implemented on any suitable processor, such as computing device 230 shown in FIGS. 2A and 2B, for example, and/or any other suitable processor, as aspects of the technology described herein are not limited in this respect.

At step 502, method 500 includes determining subsets of an acquisition period of a sensor to evaluate for patterns of noncompliance. The subsets may include any suitable subsets, as aspects of the technology described herein are not limited in this respect. As nonlimiting examples, the subsets of the acquisition period may include hours of the day, a combination of hours of the day, days, or a combination of days of the acquisition period. For example, to identify hour(s) of the day during which the subject is frequently not compliant in using the sensor (e.g., early in the morning, late in the evening, etc.), subsets of hours may be determined at act 502. As an additional or alternative example, to identify particular day(s) of an acquisition period during which the subject is frequently not compliant in using the senor (e.g., the first and/or last few days of the acquisition period, etc.), subsets of days may be determined at act 502.

At step 504, a support value is determined for each of the subsets. In some embodiments, the support value is the proportion of (a) the number of instances of said subset during which the subject was not compliant in using the sensor to (b) the total number of instances of said subset. For example, if the subsets were determined to be hours of the day, the support value for a particular subset (e.g., a particular hour of the day) may be the proportion of (a) the number of instances of the particular hour of the day during which the subject was not compliant in using the sensor during the acquisition period to (b) the total number of instances of the particular hour during the acquisition period. In some embodiments, determining whether the subject was compliant in using sensor during instances of a subset may be performed using any of the techniques described herein including at least with respect to FIG. 3A or any other suitable techniques, as aspects of the technology described herein are not limited in this respect.

In some embodiments, determining the support value for a particular subset includes subdividing said subset into multiple subdivisions and using the multiple subdivisions to determine the support value. In such embodiments, the support value may be the proportion of (a) the number of subdivisions of said subset during which the subject was not compliant in using the sensor to (b) the total number of subdivisions of said subset. For example, if the subsets were determined to be days of the acquisition period, the days may be subdivided into hours, and the support value for a particular day may be (a) the number of hours of said day during which the subject was not compliant in using the sensor to (b) the total number of hours of said day. In some embodiments, determining whether the subject was compliant in using sensor during subdivisions of a subset may be performed using any of the techniques described herein including at least with respect to FIG. 3A or any other suitable techniques, as aspects of the technology described herein are not limited in this respect.

At act 506, the support value is compared to a user-defined threshold. The threshold may include any suitable threshold as aspects of the technology described herein are not limited in this respect. For example, the threshold may be at least 0.3, at least 0.4, at least 0.5, at least 0.6, at least 0.7, at least 0.8, at least 0.9, or at least any other suitable threshold value. Additionally, or alternatively, the threshold may be at most 0.9, at most 0.8, at most 0.7, at most 0.6, at most 0.5, at most 0.4, at most 0.3, or at most any other suitable threshold value. Additionally, or alternatively, the threshold may be between 0.3 and 0.9, between 0.4 and 0.8, between 0.5 and 0.7, or between any other suitable values, as aspects of the technology described herein are not limited in this respect.

At act 508, a set of one or more subsets for which the support value is greater than or equal to the user-defined threshold is output. In some embodiments, outputting the set of subsets may be performed using the techniques described herein with respect to act 314 of method 300 shown in FIG. 3A for outputting an indication of a pattern of noncompliance. Additionally, or alternatively, the set of one or more subsets may be output using any other suitable techniques, as aspects of the technology described herein are not limited in this respect.

While the foregoing description has focused on subsets of hours of the day, or days of the acquisition period, other ways of determining subsets to evaluate for patterns of noncompliance are also possible. For example, the subsets may include days of the week (e.g., Monday, Tuesday, Wednesday, Thursday, Friday, Saturday, and Sunday). Additionally, or alternatively, the subsets may include weekdays, weekends, and/or holidays. Additionally, or alternatively, the subsets may include days during which the subset is physically located at a clinical site (e.g., for a visit) and/or days during which the subject is not physically located at a clinical site. Additionally, or alternatively, the subsets may include days on which the subject is experiencing symptoms and/or days during which the subject is not experiencing symptoms. It should be appreciated that the subsets may include any other suitable subsets, as aspects of the technology described herein are not limited in this respect.

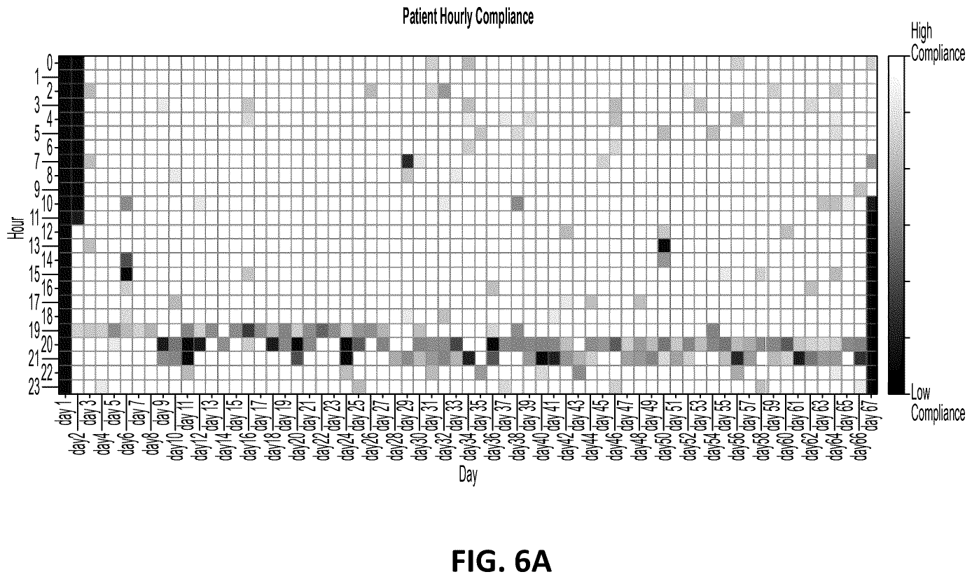

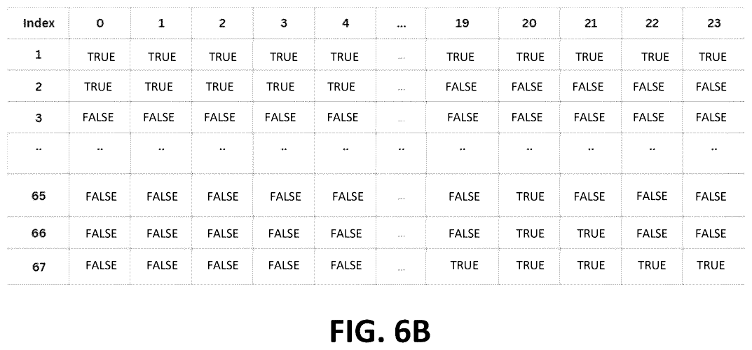

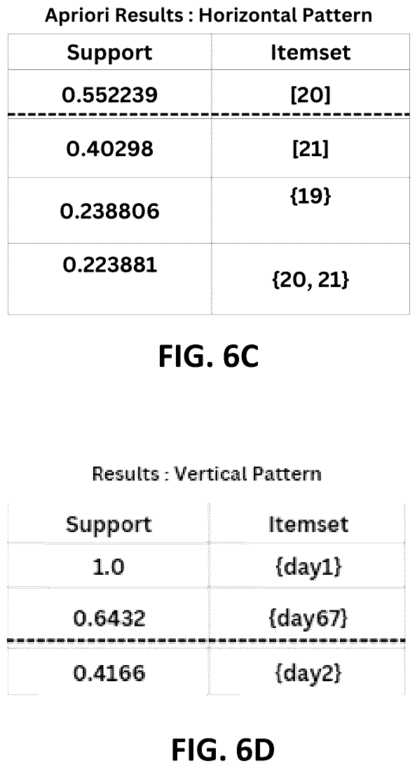

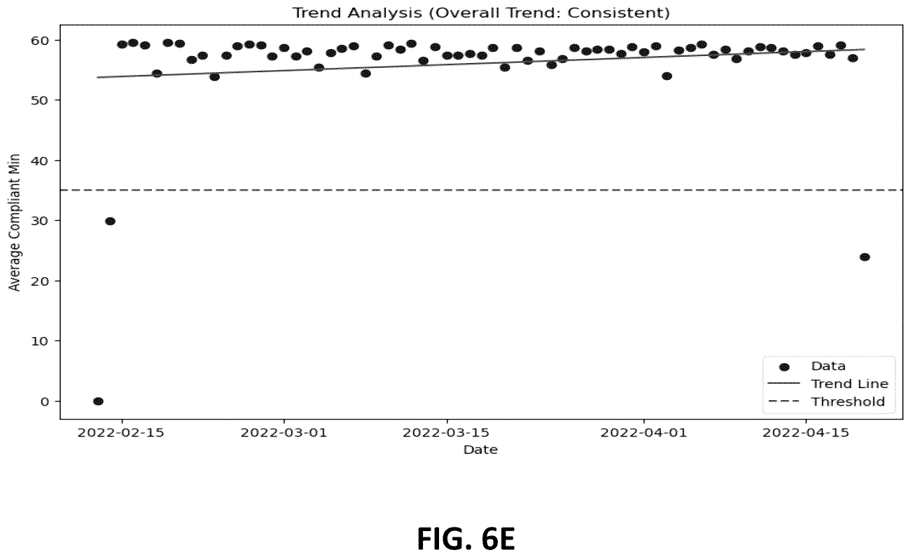

FIGS. 6A-6D show an example of determining a pattern of compliance of a first subject in using an actigraphy sensor based on indications of quality determined for the data acquired by the actigraphy sensor.

FIG. 6A is an example report indicating the degree to which the first subject was compliant in using a sensor during each hour of multiple days, according to some embodiments. The acquisition period has a duration of 67 days and is divided into 67 days, each of which is further subdivided into subperiods of one hour. Each box indicates the degree to which the first subject was compliant in using the sensor during the corresponding hour and day of the acquisition period. The degree of compliance may be calculated based on a proportion of minutes in said corresponding hour during which compliant data was received for the first subject to a total number of minutes in said corresponding hour. The higher the proportion, the higher the degree of compliance. Again, darker shading indicates low compliance, while lighter shading indicates high compliance.

FIG. 6B is an example table indicating whether or not the first subject was compliant in using the sensor during each hour of the multiple days, according to some embodiments. The rows of the table correspond to days, and the columns correspond to hours of the day. A table entry of “TRUE” indicates that the first subject was not compliant in using the sensor during a particular hour of a particular day. A table entry of “FALSE” indicates that the first subject was compliant in using the sensor during the particular hour. In some embodiments, the table may be generated based on the indications of quality shown in FIG. 6A. For example, for an hour with a quality greater than or equal to a threshold quality (e.g., 50% of the maximum quality), the corresponding table entry may be filled with a value of “FALSE”. For an hour with a quality less than the threshold, the corresponding table entry may be filled with the value of “TRUE.”

In some embodiments, the data in the table shown in FIG. 6B may be used to determine the pattern of subject noncompliance. For example, the data may be provided as input to a frequent itemset mining algorithm to determine the pattern of noncompliance. In some embodiments, the frequent itemset mining algorithm may determine support values for different portions of the acquisition period, which may be used to determine the pattern of noncompliance.

For example, determining the pattern of noncompliance may include determining support values of subsets of one or more hours of the day and using the support values to determine whether the first subject was frequently noncompliant during those hours of the day over the duration of the acquisition period. FIG. 6C shows an example table of support values determined using the data shown in FIG. 6B. As shown in FIG. 6C, hour 20 of the day has a support value of 0.552239. Intuitively, this support value indicates that in 0.552239 of the 67 days in the acquisition period, the first subject was noncompliant during hour 20. This support value is above the threshold (e.g., 0.5) indicated by the dashed line, which indicates that the first subject had a pattern of not complying in using the sensor during the 20th hour of the day. By contrast, as further shown in FIG. 6C, the first subject was typically compliant (e.g., support value below the threshold) in using the sensor during prior and subsequent hours (e.g., hours 19 and 21).

Additionally, or alternatively, determining the pattern of noncompliance may include determining support values of subsets of one or more days of the acquisition period and using the support values to determine whether the first subject was frequently noncompliant during those days of the acquisition period. FIG. 6D shows an example table of support values determined using the data shown in FIG. 6B. As shown in FIG. 6D, day 1 of the acquisition period has a support value of 1.0 (indicating the first subject was noncompliant in using the sensor during every hour of day 1), and day 67 has a support value of 0.6432 (indicating the first subject was noncompliant in 0.6432 of the hours during day 67). Both of these values are above the threshold value (e.g., 0.5), as indicated by the dashed line, which indicates that the first subject had a pattern of not complying in using the sensor during the first and 67th (last) day of the acquisition period. By contrast, as further shown in FIG. 6D, the first subject was typically compliant (e.g., support value below the threshold) in using the sensor during the day after the first day.

It should be appreciated that a simple trend analysis may not be sufficient to capture patterns of noncompliance. For example, as shown in FIG. 6E, an example trend analysis based on the data shown in FIG. 6A, shows that the first subject was generally compliant in using the sensor. Fitting a line to the compliance data would not provide any indication of the outlier compliance data points, which indicate that the first subject was not compliant in using the sensor during a couple of days of the acquisition period.

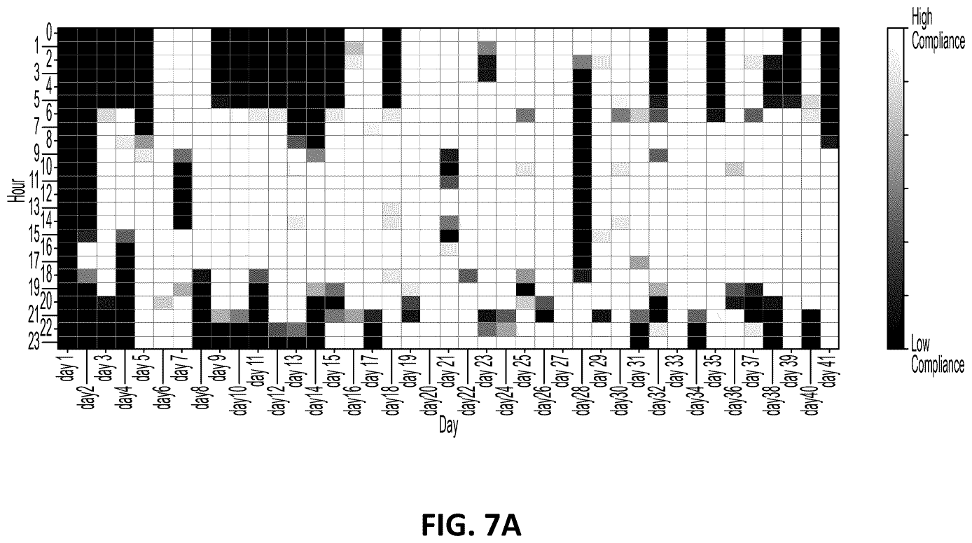

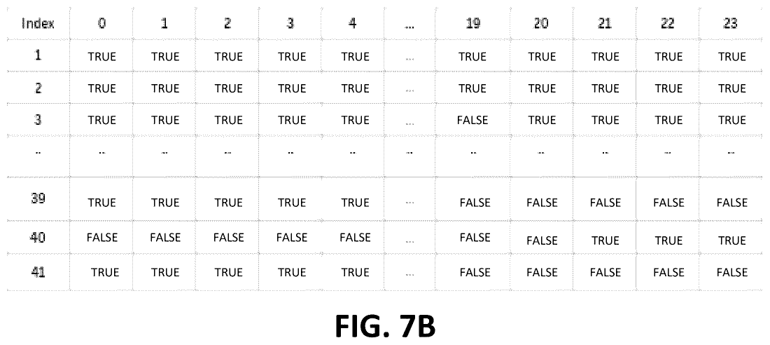

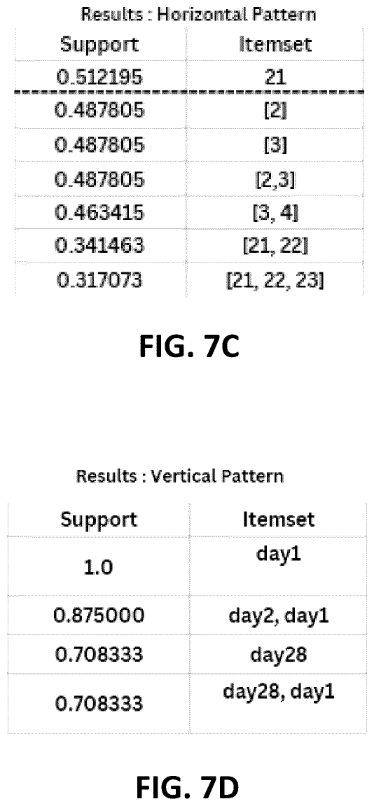

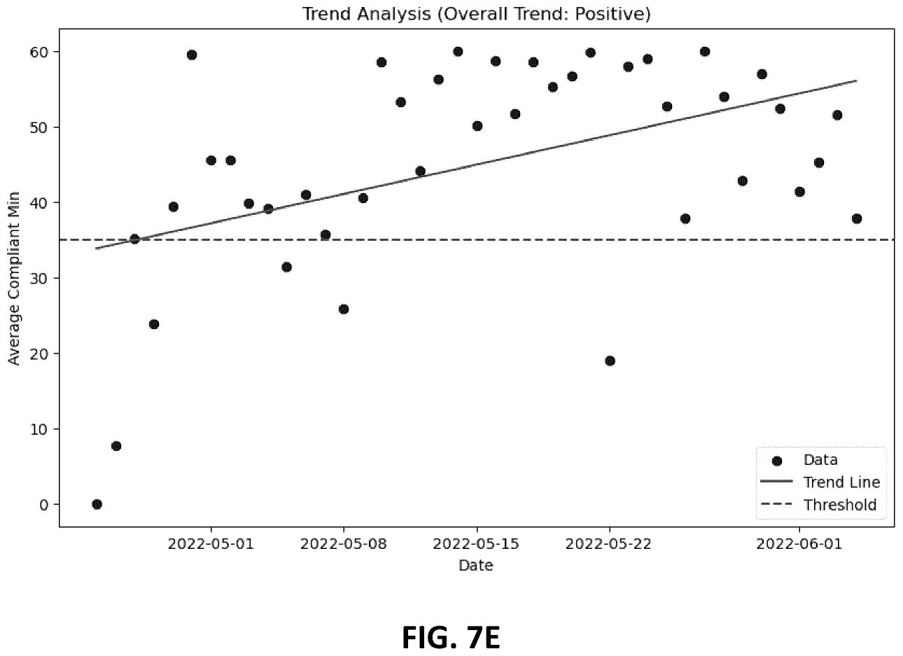

FIGS. 7A-7D show an example of determining a pattern of compliance of a second subject in using an actigraphy sensor based on indications of quality determined for the data acquired by the actigraphy sensor.

FIG. 7A is an example report indicating the degree to which the second subject was compliant in using a sensor during each hour of multiple days, according to some embodiments. The acquisition period is a duration of 41 days and is divided into 41 days which are further subdivided into subperiods of one hour. Each box indicates the degree to which the second subject was compliant in using the sensor during an hour of a specific day of the acquisition period.

FIG. 7B is an example table indicating whether or not the second subject was compliant in using the sensor during each hour of the multiple days, according to some embodiments. The rows of the table correspond to days, and the columns correspond to hours. A table entry of “FALSE” indicates that the second subject was compliant in using the sensor during a particular hour of a particular day. A table entry of “TRUE” indicates that the second subject was not compliant in using the sensor during the hour. In some embodiments, the table may be generated based on the indications of quality shown in FIG. 7A. For example, for an hour with a quality greater than or equal to a threshold quality (e.g., 50% of the maximum quality), the corresponding table entry may be filled with a value of “FALSE”. For an hour with a quality less than the threshold, the corresponding table entry may be filled with the value of “TRUE.”

In some embodiments, the data in the table shown in FIG. 7B may be used to determine the pattern of subject noncompliance. For example, the data may be provided as input to a frequent itemset mining algorithm to determine the pattern of noncompliance. In some embodiments, the frequent itemset mining algorithm may determine support values for different portions of the acquisition period, which may be used to determine the pattern of noncompliance.

For example, determining the pattern of noncompliance may include determining support values of subsets of one or more hours of the day and using the support values to determine whether the second subject was frequently noncompliant during those hours of the day over the duration of the acquisition period. FIG. 7C shows an example table of support values determined using the data shown in FIG. 7B. As shown in FIG. 7C, hour 21 of the day has a support value of 0.512195 (indicating that in 0.512195 of the 41 days in the acquisition period, the second subject was noncompliant during hour 21). This support value is above the threshold (e.g., 0.5) indicated by the dashed line, which indicates that the second subject had a pattern of not complying in using the sensor during the 21st hour of the day. By contrast, as further shown in FIG. 7C, the second subject was typically compliant (e.g., support value below the threshold) in using the sensor during prior and subsequent hours (e.g., hours 22 and 23), even when evaluated together with the compliance data obtained for hour 21.

Additionally, or alternatively, determining the pattern of noncompliance may include determining support values of subsets of one or more days of the acquisition period and using the support values to determine whether the second subject was frequently noncompliant during those days of the acquisition period. FIG. 7D shows an example table of support values determined using the data shown in FIG. 7B. As shown in FIG. 7D, day 1 of the acquisition period has a support value of 1.0 (indicating the second subject was noncompliant in using the sensor during every hour of day 1), the subset of days 1 and 2 has a support value of 0.875 (indicating the second subject was noncompliant for 0.875 of all the hours in days 1 and 2 combined), day 28 has a support value of 0.708333 (indicating the second subject was noncompliant in 0.70833 of all hours on day 28), and the subset of days 28 and 1 has a support value of 0.708333 (indicating the second subject was noncompliant for 0.70833 of all the hours on days 28 and 1 combined). All of these values are above the threshold value (e.g., 0.5), which indicates that the second subject had a pattern of not complying in using the sensor towards the beginning days and the end days of the acquisition period.

It should be appreciated that a simple trend analysis may not be sufficient to capture patterns of noncompliance. For example, as shown in FIG. 7E, an example trend analysis based on the data shown in FIG. 7A, shows that the second subject was generally compliant in using the sensor. Fitting a line to the compliance data would not provide any indication of the several outlier compliance data points, which indicate that the second subject was not compliant in using the sensor during several days of the acquisition period.

### Example 1—Example Data

As described herein, in some embodiments, data is received and/or processed to determine a quality of the data. For example, the data may include data acquired by a sensor. Examples of types of data that may be received and/or processed are described herein. It should be appreciated that these examples are not intended to be limiting, and that any other suitable types of data may be received and/or processed.

### Sensor Data

In some embodiments, data are collected from a device that measures a certain physical environment (e.g., wrist motion or temperature) in a high sampling frequency, e.g., 50 Hz. This type of data is also called high frequency point-based time series, X=(Tᵢ,Xᵢ), for i=1, . . . , m}:: a series of time-value pairs, where Tᵢ is the timestamp and Xᵢ is the signal value for ith sample point.

In some embodiments, a device may collect data from multiple sensor signals at varied preconfigured sampling frequencies. For example, a device may collect 3-axial accelerometry data at 50 Hz, electrodermal activity (EDA) data at 4 Hz, and body temperature data at 1 Hz. In some embodiments, the sensor signals are collected in a nonstop 24*7 fashion throughout an entire study, which may run between weeks to months.

### Scored Data or Digital Biomarkers

In some embodiments, in addition to raw sensor signals, a device may process the sensor data and derive digital biomarkers (dBMs) from it. For example, heart rate and blood volume pulse can be derived from raw photoplethysmography (PPG) sensor signal. Derived dBMs may be at a lower resolution than the sensor signal.

Electronic-Reported Outcomes (ePROs)

In some embodiments, Electronic Patient-Reported Outcomes are gathered from study participants for comparison with digital biomarkers (dBM).

### Temporal Events

Contrary to point-based time-series, temporal events can be viewed as interval-based timeseries with a start and end timestamp for each event. One example is a patient-reported migraine event with a start and end timestamp, along with reported pain levels during the migraine on a scale of 10. This interval-based variable can be expressed as W={(Tₛᵢ, Tₑᵢ, Yᵢ), for i=1, . . . , m}:: a set of time intervals, with Tₛᵢ and Tₑᵢ stand for the onset and offset timestamps, and Yᵢ is the binary indication of the event for ith interval.

### Example 2—Example System for Receiving and Processing Data

As described herein, one or more computing devices may be configured to receive data (e.g., from a sensor) and process the data. For example, the computing device(s) may be configured to process the data, using software, to determine a quality of the data and/or a subset of the data. An example system for receiving and processing data is described herein. It should be appreciated that these examples are not intended to be limiting, and that any other suitable techniques may be used to receive and process data.

### Data Sources

In some embodiments, two types of data may be received from study participants: high-resolution raw sensor signals from wearable sensors (e.g., wrist-worn devices, chest patches, and foot insole sensors) and ePROs submitted via mobile applications or web forms. In addition, metadata may be obtained from Clinical Research Organizations (CROs), such as mappings between sensor devices to participants, participant visit schedule information (a typical study or trial consists of a sequence of visits, with each visit spanning several days), and treatment group or cohort placement (e.g., placebo vs. treatment with specific medicine dose).

### Data Transfer Tools

In some embodiments, data transfer tools enable data to be received in a digital data platform (DDP) at scale, and they interface with storage infrastructure without involving intermediate data staging storage or area. From the perspective of data transfer cadence, a secure file transfer protocol, such as Amazon Web Services secure shell file transfer protocol (AWS SFTP) services, may enable data transfer in batches. Furthermore, services, such as AWS Kinesis services (i.e., Kinesis Data Streams and Firehose) can be leveraged to receive data streams (e.g., real-time data streams) directly from sensors. In addition, various sync tools may also allow the DDP to pull data from external sources at configurable cadences, allowing extra flexibility.

### Storage Infrastructure

In some embodiments, storage infrastructure interfaces with data transfer tools through preconfigured listeners and triggers, with which data flows (e.g., automatically) in upon arrival. The DDP may allow data to land in fit-for-purpose storage components by three metrics: (1) I/O performance; (2) interfaces (e.g., APIs) available to access data and interface with other storage/computing components; and (3) the cost. Based on this principle, the infrastructure, in some embodiments, has four categories.

The first category may include a data lake targeting raw data like sensor signals, composed of Parallel File Systems (PFS, e.g., Lustre) in an on-premise High Performance Computing (HPC) environment and storage buckets and tapes in the cloud (e.g., AWS S3 for hot data and Glacier for cold data).

Second, for structured data or DataFrame alike that involve read access, table viewers (e.g., AWS Glue tables) read underlying data lakes in the meantime with additional layers for performance optimization and SQLbased view/query interface for data access.

The third category may include application-specific high-performance data structures and data stores. For instance, sensor signals and mission-critical data may be converted into Elasticsearch's internal data structure, i.e., Lucene indices, to allow near real-time aggregation and on-the-fly data query.

Fourth, dedicated relational databases may be leveraged for structured data that includes a write operation or whose query performance cannot be guaranteed by the table view.

### Computing Infrastructure

In some embodiments, computing infrastructure serves as a heavy-duty computing engine. To support automation, in some embodiments, listeners and triggers are preconfigured to launch downstream computation once upstream storage feeds data.

In this example, the computing engine is partitioned into two logical layers. The first layer may include a layer of computing frameworks that sits on the bottom. This layer may support application-agnostic data handling and processing at scale. For example, crawlers and data catalogs, along with lambda event listeners, produce table viewers for structure data; Spark conducts generic data format conversion (e.g., CSV to more performant Apache parquet) in parallel; and custom scripts/frameworks support scalable data processing in HPC environment.

The second layer may include a layer of business logic-specific pipelines that sits on the top. This layer may leverage the underlying computing frameworks layer to run processing at scale. For example, an accelerometry sensor data analysis pipeline may be developed on top of open-source algorithms (e.g., GGIR and UKBiobank) and deployed (e.g., through Spark) in the cloud along with parallel array jobs. Additional algorithms may be deployed for further processing.

### Frontend Portal

In some embodiments, interactive and iterative visual queries enable the concept of “humanin-the-loop” analytics and are fulfilled through a web-based portal, which contains dashboards. For example, each dashboard may have multiple filters and operators that refine visual queries iteratively. In addition to dashboards, the DDP may include practical information such as study portfolios, technical manuals, data processing pipelines, and pointers to internal GitHub repositories.

### APIs and Analytics Environment

In some embodiments, APIs and software development kits (SDKs) target different data access and query needs, including metadata query, raw data query, dynamic data aggregation, data ingestion, and business specific utility APIs.

In some embodiments, a set of APIs and/or SDKs are used to facilitate data access. For example, a digital data platform may integrate APIs (e.g., AWS S3 and AWS Athena) to enable SQL data queries and direct file loading into the analytics environment. AWS S3 is a highly scalable, durable, and secure object storage cloud service. AWS Athena is a serverless query service backed by AWS S3.

In some embodiments, a search engine, such as AWS-managed Elasticsearch, for example, enables near real-time search and data aggregation. Elasticsearch is a data store, search, and analytics engine based on Lucene. The AWS-managed service allows on-demand up-scaling of Elasticsearch as the data volume increases.

### Data Flow

In some embodiments, sensor and ePROs data arrive at the storage layer through high-performant transfer tools and services. Once data lands, in some embodiments, one or more processing pipelines are triggered, either in parallel (when there is no dependency) or in a particular order (i.e., chained pipelines when the sequence matters). One example is a spark pipeline for raw data cleaning and quality checking, followed by concurrent feature extraction pipelines, each of which handles a specific sensor data type. For example, an accelerometry data processing pipeline may involve one or more modules including, but not limited to: (1) calibration to local gravity; (2) resampling; (3) gravity removal; (4) noise removal; (5) data segmentation; and (6) features calculation. In some embodiments, once features from the data channels (e.g., accelerometer, electrodermal activity, and temperature) are ready, a feature aggregation pipeline launches to perform aggregation at different levels, from hourly, daily, weekly, or per visit in the study.

### Example 3—Example Visualizations

As described herein, in some embodiments, the techniques for determining a quality of a signal acquired by a sensor include outputting an indication of the quality. Examples for outputting visualizations of data and indications of quality of the data are described herein. It should be appreciated that these examples are not intended to be limiting, and that any other suitable techniques for generating an output may be implemented, as aspects of the technology are not limited in this respect.

In some embodiments, an integration tool, such as AWS Kibana, is used to leverage stored data (e.g., data stored in AWS Elasticsearch). AWS Kibana can create customized visualizations with near real-time interactivity. Multiple data types associated with clinical trials can be viewed in various formats, including, for example, time-series plots, histograms, heatmaps, and data tables. In some embodiments, during connected clinical trials, digital data are continuously ingested, so, based on the data structure and purpose, visualizations can be rendered to help track the progression and quality of digital data in the trials.

In some embodiments, visualizations are tailored to display accurate data based on time filters or applied queries. As a nonlimiting example, a visualization may be generated for a biosensor signal. Such data may include, for example, raw accelerometer sensor data with a sampling frequency of 50 Hz (e.g., a data point every 20 milliseconds). Accordingly, in the example, the visualization may be capable of showing data every 20 milliseconds and may be capable of zooming in and out based on a particular level of detail or pattern to be identified. In some embodiments, with an integration tool such as AWS Kibana, an existing AWS Elasticsearch indexed DataFrame can be selected as the data source. Additionally, an aggregation method and type of plot may be selected. Nonlimiting examples of aggregations include average, maximum, minimum, percentile, standard deviation, sum, and variance. Depending on the zoom level, the data aggregation may, in some embodiments, dynamically update to an interval that suits the visualization's date and time range (e.g., 1 second, 1 hour, 1 day).

In some embodiments, temporal events are plotted. Plotting events may be useful for understanding and comparing the ground truth of reported symptoms and events to sensor data. For example, a step-line plot allows for the translation of data with timestamps and labels for each timestamp (i.e., the start and end timestamp of an event) into a time-series plot that shows the different categories of events. Additionally, or alternatively, for events with a scale rating, changes in the reported rankings can be seen at indicated time points in the event.

In some embodiments, derived features, such as step count, sleep minutes, or heart rate, for example, can be viewed in a time-series bar plot. This may be similar to a time-series plot for viewing accelerometer data. Additionally, or alternatively, other derived information in connected clinical trials may be viewed, including, but not limited to, a matrix of data compliance percentages to track digital data quality throughout the trials and participants' geo-locations in decentralized trials.

In some embodiments, the visualizations can be shown independently (e.g., separately) or multiple visualizations can be viewed simultaneously at a given time.

### Example 4—Example Techniques for Determining Data Quality

As described herein, including at least with respect to FIG. 1A-FIG. 4D, data acquired by a sensor may be processed to determine a quality of the data. Examples of determining a quality of sensor data are described herein. It should be appreciated that these examples are not intended to be limiting, and that any other suitable techniques for determining a quality of data may be implemented, including those described herein with respect to FIG. 1A-FIG. 4D.

### Validity Check

In some embodiments, the sensor data is assessed to filter out invalid data. For example, it is possible to determine expected number of valid data points based on the pre-configured sampling frequency. Invalid values can be filtered out using the valid value range to get valid data coverage, i.e., coverage of valid data points.

Additionally, or alternatively, in some embodiments, since raw sensor signal directly correlates with derived dBMs, a validity check can be performed against the two independently, and then their valid data coverage can be aligned to check the consistency. In some embodiments, device incident events may be overlayed to better understand the root cause of observed issues. Invalid data may be dropped.

### Non-Use and Clipping Detection

In some embodiments, although data points may be collected initially at a high resolution, e.g., 50 Hz sampling frequency, the processing is conducted on aggregated values (e.g., 1 or 5 second short subperiods (epochs) or 15 minutes long subperiods (epochs)).

In some embodiments, accelerometer data is used to determine whether a subject was using their sensor during a particular subperiod. In some embodiments, accelerometer non-wear time is estimated based on the standard deviation and the value range of the raw data from each accelerometer axis. As an example, classification may be done per 15-minute subperiod based on the characteristics of a 60-minute acquisition period centered at these 15-minute subperiods. A subperiod may be classified as non-wear time if the standard deviation of the 60-minute acquisition period is less than 13.0 mg (1 mg=0.0098 m·s-2) and the value range of the 60-minute window is less than 50 mg for at least two out of the three accelerometer axes.

In some embodiments, accelerometer data may also be screened for “clipping.” As an example, if more than 50% of data points in a 15-minute time window (subperiod) are close to the maximal dynamic range of the sensor (e.g., 7.5g), the corresponding subperiod may be considered potentially corrupt as the majority are of extreme value.

In some embodiments, the techniques include determining whether data corresponding to a particular subperiod is valid based on whether the subject was using the sensor and/or whether there was clipping. Table 1 shows an example of a validity table for different subperiods. Values in the “Non-Use Score” column range between 0-3, representing the sum of the three independent scores from the x, y, and z-axis, respectively. An axis earns a score of 1 when detected as non-use and 0 otherwise. Moreover, for the “Clipping Score” column, a value of 1 means corrupted data are detected and 0 otherwise. Based on these two scores, a third column, called “is Valid,” can be derived to indicate data validity in a subperiod, where the data is considered to valid if the non-use score is less than or equal to one and where the clipping score is zero.

TABLE 1
Validity Table.

| Timestamp | Non-Use Score | Clipping Score | isValid |
|---|---|---|---|
| 2021 Sep. 15 | 3 | 0 | False |
| 2021 Sep. 15 | 2 | 0 | False |
| 2021 Sep. 15 | 1 | 0 | True |
| . . . | . . . | . . . | . . . |
| 2021 Sep. 18 | 0 | 1 | False |
| 2021 Sep. 18 | 0 | 0 | True |

In some embodiments, the data can be processed to determine data coverage at an hourly level. For example, this may include, starting from the validity table, applying a filter on the “isValid” flag and then grouping the results by subject, date, and hour to get the data coverage in minutes on an hourly level. In some embodiments, by grouping and counting records in each hour, hourly data coverage can be derived since each long period lasts 15 minutes. The hourly data coverage may be the source for data coverage reporting at the finest granularity.

In some embodiments, the data can be processed to determine coverage at an hourly level. For example, the hourly data coverage can be aggregated through summation over days to have daily level data coverage. In addition, in some embodiments, intraday coverage may be derived.

In some embodiments, the data can be processed to determine extended data coverage with external mappings. For example, the data coverage can be extended with additional mappings such as mapping between subjects and sites/visits, as reported from the clinical operation site. These extra fields may allow analysis-specific filtering and aggregation, e.g., to find out which participants have sufficient data and set up individual baselines. For example, such data may be used to identify subjects with at least three valid days (>=20 hours of data for a day to be qualified as a valid day) during a pre-treatment visit.

### Example 5—Example Indication of Quality

As described herein, in some embodiments, the techniques for determining a quality of a signal acquired by a sensor include outputting an indication of the quality. Examples indications of determined quality are described herein. It should be appreciated that these examples are not intended to be limiting, and that any other suitable indications of quality may be implements.

### Minute-by-Minute Quality Map for a Day

Examining signals on a minute level can help to identify the minutes where a device may have intermittent connectivity, or more minor issues can be identified and further inspected.

### Hour-by-Hour Quality Map for a Trial

In some embodiments, the hourly level aggregation is used to configure the day level plot. An hour-by-hour quality map shows data coverage for each hour across all study days. This type of visualization allows for the evaluation of compliance trends for a patient that may persist during certain hours of each day. For example, a patient may take off a wearable device to charge the battery for a couple of hours each day, which may result in missing data. For example, FIG. 4D shows that, on several days of the trial period, the patient did not use the device for an hour or so in the middle of the day.

### Day-by-Day Population-Level Quality Map for a Trial

In some embodiments, plotting data quality for all hours, days and participants in a study yields the observation of data quality patterns. Such a study-level visualization can help to gain insights into the overall data quality at the population level and the compliance trends at the participant level throughout the trials.

### Compliant Days Throughout a Trial

In some embodiments, it is also useful to view the number of compliant days throughout the study, with a definition of compliance dependent on a study's protocol. One can recognize device-wearing patterns by plotting the number of patients compliant daily in a given study (e.g., as shown in FIG. 3C).

Techniques operating according to the principles described herein may be implemented in any suitable manner. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

Further, some techniques described above comprise acts of storing information (e.g., data and/or instructions) in certain ways for use by these techniques. In some implementations of these techniques—such as implementations where the techniques are implemented as computer-executable instructions—the information may be encoded on a computer-readable storage media. Where specific structures are described herein as advantageous formats in which to store this information, these structures may be used to impart a physical organization of the information when encoded on the storage medium. These advantageous structures may then provide functionality to the storage medium by affecting operations of one or more processors interacting with the information; for example, by increasing the efficiency of computer operations performed by the processor(s).

In some, but not all, implementations in which the techniques may be embodied as computer-executable instructions, these instructions may be executed on one or more suitable computing device(s) operating in any suitable computer system, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more Field-Programmable Gate Arrays (FPGAs) for carrying out the techniques described herein, or any other suitable system.

A computing device may comprise at least one processor, a network adapter, and computer-readable storage media. A computing device may be, for example, a desktop or laptop personal computer, a personal digital assistant (PDA), a smart mobile phone, a server, or any other suitable computing device. A network adapter may be any suitable hardware and/or software to enable the computing device to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media may be adapted to store data to be processed and/or instructions to be executed by processor. The processor enables processing of data and execution of instructions. The data and instructions may be stored on the computer-readable storage media.

A computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

While various embodiments have been described, it will be apparent to those of ordinary skill in the art that many more embodiments and implementations are possible. Accordingly, the embodiments described herein are examples, not the only possible embodiments and implementations. Furthermore, the advantages described above are not necessarily the only advantages, and it is not necessarily expected that all of the described advantages will be achieved with every embodiment.

Various aspects are described in this disclosure, which include, but are not limited to, the following aspects:

1. A method, comprising: using one or more processors to perform: receiving, from a plurality of actigraphy sensors associated with a plurality of subjects, data acquired by the plurality of actigraphy sensors during an acquisition period, wherein the data is indicative of signals associated with bodily movement of the plurality of subjects, the receiving comprising: receiving, from a first actigraphy sensor of the plurality of actigraphy sensors associated with a first subject of the plurality of subjects, first data acquired by the first actigraphy sensor, wherein the first data is indicative of signals associated with bodily movements of the first subject; dividing the acquisition period into a plurality of subperiods; aggregating at least some of the data into a plurality of subsets of data, the at least some of the data including the first data, wherein each of the plurality of subsets of data comprises data acquired during a different subperiod of the plurality of subperiods; determining, for each particular subperiod of the plurality of subperiods, a quality of the subset of data acquired during the particular subperiod, the determining comprising: determining whether the first subject of the plurality of subjects was compliant in using the first actigraphy sensor of the plurality of actigraphy sensors during the particular subperiod; and outputting an indication of the determined quality of at least one subset of data of the plurality of subsets of data.

2. The method of aspect 1, wherein determining whether the first subject was compliant in using the first actigraphy sensor comprises determining whether the first subject was using the first actigraphy sensor.

3. The method of aspect 2, wherein the subset of data acquired during the particular subperiod is indicative of an acceleration of a portion of a body of the first subject during the particular subperiod, and wherein determining whether the first subject was using the first actigraphy sensor during the particular subperiod comprises: comparing the acceleration of the portion of the body of the first subject to an acceleration threshold; and based on a result of the comparing, determining whether the first subject was using the first actigraphy sensor during the particular subperiod.

4. The method of any of aspects 1-3, wherein determining the quality of the subset of data acquired during the particular subperiod further comprises determining whether the first actigraphy sensor malfunctioned during the particular subperiod.

5. The method of aspect 4, wherein determining whether the first actigraphy sensor malfunctioned during the particular subperiod comprises determining a proportion of the subset of data that is equal to a maximum or a minimum output value of the first actigraphy sensor, and wherein the indication of the determined quality of the at least one subset of data includes an indication of the determined proportion of the subset of data.

6. The method of aspect 4, wherein determining whether the first actigraphy sensor malfunctioned comprises determining whether the subset of data includes values excluded from an output range of the first actigraphy sensor.

7. The method of any of aspects 1-6, wherein the plurality of subperiods is a plurality of first subperiods, each subperiod of the plurality of first subperiods being of a first duration, wherein the method further comprises: dividing the acquisition period into a plurality of second subperiods, each second subperiod being of a second duration different from the first duration; aggregating at least some of the data into a plurality of second subsets of data, wherein each second subset of data of the plurality of second subsets of data comprises data acquired during a different second subperiod of the plurality of second subperiods; determining, for each particular second subperiod of the plurality of second subperiods, a quality of the second subset of data acquired during said particular second subperiod; and outputting an indication of the determined quality of at least one second subset of data of the plurality of second subsets of data.

8. The method of aspect 7, wherein the first duration is longer than the second duration.

9. The method of any of aspects 1-8, wherein each of the plurality of subjects is participating in a clinical study, the clinical study having one or more clinical study sites, wherein each site of the one or more clinical study sites is associated with one or more subjects of the plurality of subjects, and wherein the data acquired by the plurality of actigraphy sensors comprises data associated with the clinical study.

10. The method of aspect 9, further comprising: determining study-level compliance of the plurality of subjects in using the plurality of actigraphy sensors during the acquisition period; and outputting a report indicating the study-level compliance.

11. The method of aspect 9, wherein the at least some of the data consists of data associated with a particular site of the one or more sites of the clinical study, the particular site being associated with a respective one or more subjects of the plurality of subjects, the method further comprising: determining site-level compliance of the respective one or more subjects in using a respective one or more actigraphy sensors of the plurality of actigraphy sensors during the acquisition period, and outputting a report indicating the site-level compliance.

12. The method of any of aspects 1-11, wherein outputting the indication of the determined quality of the at least one subset of data comprises: generating a graphical user interface; and displaying a visual indication through the graphical user interface.

13. The method of any of aspects 1-12, further comprising: prior to dividing the acquisition period into the plurality of subperiods, receiving, from a user, an indication of a duration of each subperiod of the plurality of subperiods, wherein dividing the acquisition period into the plurality of subperiods comprises dividing the acquisition period according to the received indicated duration, such that each subperiod of the plurality of subperiods has a duration that corresponds to the received indicated duration.

14. The method of any of aspects 1-13, further comprising: prior to determining the quality of the subset of data acquired during said particular subperiod, receiving user input indicating one or more criteria for determining the quality of the subset of data acquired during said particular subperiod, wherein determining the quality of the subset of data acquired during said particular subperiod comprises determining the quality of the subset of data according to the received user input indicating the one or more criteria.

15. The method of any of aspects 1-14, wherein determining the quality of the at least some of the data comprises determining the quality in real-time.

16. The method of any of aspects 1-15, further comprising: determining, based on the quality determined for each subperiod of the plurality of subperiods, a pattern of non-compliance of one or more subjects of the plurality of subjects in using a respective one or more actigraphy sensors of the plurality of actigraphy sensors; and outputting an indication of the determined pattern of non-compliance.

17. The method of aspect 16, further comprising: generating, based on the determined pattern of non-compliance, an output prompting the one or more subjects regarding how to use the respective one or more actigraphy sensor.

18. The method of any of aspects 1-17, wherein a duration of the acquisition period is a plurality of days, and wherein the method further comprises: identifying, based on the quality determined for each subperiod of the plurality of subperiods, one or more days of the plurality of days during which the first subject was not compliant in using the first actigraphy sensor; and outputting an indication of the one more days during which the first subject was not compliant in using the first actigraphy sensor.

19. The method of aspect 18, wherein identifying the one or more days during which the first subject was not compliant in using the first actigraphy sensor comprises: determining, for each respective day of the plurality of days, a proportion of a number of hours during which the first subject was not compliant in using the first actigraphy sensor to a total number of hours in said respective day; determining a subset of days for which the proportion is greater than or equal to a threshold; and identifying the determined subset of days as the one or more days during which the first subject was not compliant in using the first actigraphy sensor.

20. The method of any of aspects 1-19, wherein dividing the acquisition period into a plurality of subperiods comprises dividing the acquisition period into a plurality of days, wherein each day is further subdivided into a plurality of hours, and wherein the method further comprises: identifying, based on the quality determined for each subperiod of the plurality of subperiods, one or more hours of the plurality of hours over the plurality of days during which the first subject was not compliant in using the first actigraphy sensor; and outputting an indication of the one or more hours during which the first subject was not compliant in using the first actigraphy sensor.

21. The method of aspect 20, wherein identifying the one or more hours over the plurality of days during which the first subject was not compliant in using the first actigraphy sensor comprises: determining, for each respective hour of the plurality of hours, a proportion of a number of days of the plurality of days during which the first subject was not compliant in using the first actigraphy sensor to a total number of days in said plurality of days; determining a subset of hours for which the proportion is greater than or equal to a threshold; and identifying the determined subset of hours as the one or more hours during which the first subject was not compliant in using the first actigraphy sensor.

22. The method of any one of aspects 1-21, further comprising: outputting instructions prompting at least one subject of the plurality of subjects regarding how to use at least one actigraphy sensor of the plurality of actigraphy sensors.

23. The method of any one of aspects 1-22, further comprising: determining, for each particular subset of data of the plurality of subsets of data, whether the quality determined for the particular subset of data satisfies at least one criterion; when the particular subset satisfies the at least one criterion, using the particular subset of data to predict a health outcome for at least one subject of the plurality of subjects; and when the particular subset does not satisfy the at least one criterion, refraining from using the particular subset of data to predict the health outcome for the at least one subject.

24. The method of any of aspects 1-23, wherein receiving the data acquired by the plurality of actigraphy sensors comprises receiving the data at least once per hour during the acquisition period.

25. The method of any of aspects 1-24, wherein the acquisition period is at least 20 hours.

26. The method of aspect 25, wherein the acquisition period is a plurality of days.

27. A system comprising a memory storing instructions, and a processor configured to execute the instructions to perform the method of any of aspects 1-26.

28. A non-transitory computer-readable media comprising instructions that, when executed by one or more processors on a computing device, are operable to cause the one or more processors to execute the method of any of aspects 1-26.

What is claimed is:

1. A method for ensuring quality of data collected from a plurality of actigraphy sensors associated with a plurality of subjects participating in a clinical study, comprising:
using one or more processors to perform:
receiving, from the plurality of actigraphy sensors, data acquired by the plurality of actigraphy sensors during an acquisition period, wherein the data is indicative of signals associated with bodily movement of the plurality of subjects, the receiving comprising:
receiving, from a first actigraphy sensor of the plurality of actigraphy sensors associated with a first subject of the plurality of subjects, first data acquired by the first actigraphy sensor, wherein the first data is indicative of signals associated with bodily movements of the first subject;
dividing the acquisition period into a plurality of subperiods;
aggregating at least some of the data into a plurality of subsets of data, the at least some of the data including the first data, wherein each of the plurality of subsets of data comprises data acquired during a different subperiod of the plurality of subperiods;
determining, for each particular subperiod of the plurality of subperiods, a quality of the subset of data acquired during the particular subperiod, the determining comprising:
determining whether the first subject of the plurality of subjects was compliant in using the first actigraphy sensor of the plurality of actigraphy sensors during the particular subperiod;
determining, for each particular subset of data of the plurality of subsets of data, whether the quality determined for the particular subset of data satisfies at least one criterion;
when the particular subset satisfies the at least one criterion, using the particular subset of data to predict a health outcome for at least one subject of the plurality of subjects; and
when the particular subset does not satisfy the at least one criterion, refraining from using the particular subset of data to predict the health outcome for the at least one subject.

2. The method of claim 1,
wherein determining whether the first subject was compliant in using the first actigraphy sensor comprises determining whether the first subject was using the first actigraphy sensor.

3. (canceled)

4. The method of claim 1,
wherein determining the quality of the subset of data acquired during the particular subperiod further comprises determining whether the first actigraphy sensor malfunctioned during the particular subperiod.

5. The method of claim 4,
wherein determining whether the first actigraphy sensor malfunctioned during the particular subperiod comprises determining a proportion of the subset of data that is equal to a maximum or a minimum output value of the first actigraphy sensor, and
wherein the indication of the determined quality of the at least one subset of data includes an indication of the determined proportion of the subset of data.

6. The method of claim 4, wherein determining whether the first actigraphy sensor malfunctioned comprises determining whether the subset of data includes values excluded from an output range of the first actigraphy sensor.

7. The method of claim 1,
wherein the plurality of subperiods is a plurality of first subperiods, each subperiod of the plurality of first subperiods being of a first duration,
wherein the method further comprises:
dividing the acquisition period into a plurality of second subperiods, each second subperiod being of a second duration different from the first duration;
aggregating at least some of the data into a plurality of second subsets of data, wherein each second subset of data of the plurality of second subsets of data comprises data acquired during a different second subperiod of the plurality of second subperiods;
determining, for each particular second subperiod of the plurality of second subperiods, a quality of the second subset of data acquired during said particular second subperiod; and
outputting an indication of the determined quality of at least one second subset of data of the plurality of second subsets of data.

8. (canceled)

9. The method of claim 1, wherein the clinical study has one or more clinical study sites, wherein each site of the one or more clinical study sites is associated with one or more subjects of the plurality of subjects, the method further comprising
determining study-level compliance of the plurality of subjects in using the plurality of actigraphy sensors and outputting a report indicating the study-level compliance; and
determining a level of site-level compliance for each of the one or more clinical study sites and outputting a report indicating at least one of the determined levels of site-level compliance.

10-13. (canceled)

14. The method of claim 1, further comprising:
prior to determining the quality of the subset of data acquired during said particular subperiod, receiving user input indicating one or more criteria for determining the quality of the subset of data acquired during said particular subperiod,
wherein determining the quality of the subset of data acquired during said particular subperiod comprises determining the quality of the subset of data according to the received user input indicating the one or more criteria.

15. (canceled)

16. The method of claim 1, further comprising:
determining, based on the quality determined for each subperiod of the plurality of subperiods, a pattern of non-compliance of one or more subjects of the plurality of subjects in using a respective one or more actigraphy sensors of the plurality of actigraphy sensors; and
outputting an indication of the determined pattern of non-compliance.

17. The method of claim 16, further comprising:
generating, based on the determined pattern of non-compliance, an output prompting the one or more subjects regarding how to use the respective one or more actigraphy sensor.

18. The method of claim 1, wherein a duration of the acquisition period is a plurality of days, and wherein the method further comprises:
identifying, based on the quality determined for each subperiod of the plurality of subperiods, one or more days of the plurality of days during which the first subject was not compliant in using the first actigraphy sensor, said identifying comprising:
determining, for each respective day of the plurality of days, a proportion of a number of hours during which the first subject was not compliant in using the first actigraphy sensor to a total number of hours in said respective day,
determining a subset of days for which the proportion is greater than or equal to a threshold, and
identifying the determined subset of days as the one or more days during which the first subject was not compliant in using the first actigraphy sensor; and
outputting an indication of the one more days during which the first subject was not compliant in using the first actigraphy sensor.

19. (canceled)

20. The method of claim 1, wherein dividing the acquisition period into a plurality of subperiods comprises dividing the acquisition period into a plurality of days, wherein each day is further subdivided into a plurality of hours, and wherein the method further comprises:
identifying, based on the quality determined for each subperiod of the plurality of subperiods, one or more hours of the plurality of hours over the plurality of days during which the first subject was not compliant in using the first actigraphy sensor; and
outputting an indication of the one or more hours during which the first subject was not compliant in using the first actigraphy sensor.

21. The method of claim 20, wherein identifying the one or more hours over the plurality of days during which the first subject was not compliant in using the first actigraphy sensor comprises:
determining, for each respective hour of the plurality of hours, a proportion of a number of days of the plurality of days during which the first subject was not compliant in using the first actigraphy sensor to a total number of days in said plurality of days;
determining a subset of hours for which the proportion is greater than or equal to a threshold; and
identifying the determined subset of hours as the one or more hours during which the first subject was not compliant in using the first actigraphy sensor.

22. The method of claim 1, further comprising:
outputting instructions prompting at least one subject of the plurality of subjects regarding how to use at least one actigraphy sensor of the plurality of actigraphy sensors.

23-26. (canceled)

27. A system for ensuring quality of data collected from a plurality of actigraphy sensors associated with a plurality of subjects participating in a clinical study, the system comprising:
a memory storing instructions, and
a processor configured to execute the instructions to:
receive, from the plurality of actigraphy sensors, data acquired by the plurality of actigraphy sensors during an acquisition period, wherein the data is indicative of signals associated with bodily movement of the plurality of subjects, the receiving comprising:
receiving, from a first actigraphy sensor of the plurality of actigraphy sensors associated with a first subject of the plurality of subjects, first data acquired by the first actigraphy sensor, wherein the first data is indicative of signals associated with bodily movements of the first subject;
divide the acquisition period into a plurality of subperiods;
aggregate at least some of the data into a plurality of subsets of data, the at least some of the data including the first data, wherein each of the plurality of subsets of data comprises data acquired during a different subperiod of the plurality of subperiods;
determine, for each particular subperiod of the plurality of subperiods, a quality of the subset of data acquired during the particular subperiod, the determining comprising:
determining whether the first subject of the plurality of subjects was compliant in using the first actigraphy sensor of the plurality of actigraphy sensors during the particular subperiod;
determine, for each particular subset of data of the plurality of subsets of data whether the quality determined for the particular subset of data satisfies at least one criterion;
when the particular subset satisfies the at least one criterion, using the particular subset of data to predict a health outcome for at least one subject of the plurality of subjects; and
when the particular subset dos not satisfy the at last one criterion, refraining from using the particular subset to predict the health outcome for the at least one subject.

28. (canceled)

29. The system of claim 27, wherein the system further comprises the plurality of actigraphy sensors.

30. The system of claim 27, wherein determining whether the first subject was compliant in using the first actigraphy sensor comprises determining whether the first subject was using the first actigraphy sensor.

31. The system of claim 27, wherein determining the quality of the subset of data acquired during the particular subperiod further comprises determining whether the first actigraphy sensor malfunctioned during the particular subperiod.

32. The system of claim 27, wherein the plurality of subperiods is a plurality of first subperiods, each subperiod of the plurality of first subperiods being of a first duration, wherein the processor is further configured to execute the instructions to:
divide the acquisition period into a plurality of second subperiods, each second subperiod being of a second duration different from the first duration;
aggregate at least some of the data into a plurality of second subsets of data, wherein each second subset of data of the plurality of second subsets of data comprises data acquired during a different second subperiod of the plurality of second subperiods;
determine, for each particular second subperiod of the plurality of second subperiods, a quality of the second subset of data acquired during said particular second subperiod; and
output an indication of the determined quality of at least one second subset of data of the plurality of second subsets of data.

33. The system of claim 27, wherein the processor is further configured to execute the instructions to:
determine, based on the quality determined for each subperiod of the plurality of subperiods, a pattern of non-compliance of one or more subjects of the plurality of subjects in using a respective one or more actigraphy sensors of the plurality of actigraphy sensors; and
output an indication of the determined pattern of non-compliance
